SEQUENCE LISTING (1 sequence)

SEQ ID NO: 1 - DNA, 20 bp, synthetic construct
  source 1..20: mol_type = other DNA
gcagaggtgaagcgaagtgc

(19) United States
(12) Patent Application Publication
(10) Pub. No.: US 2025/0215431 A1
(43) Pub. Date: Jul. 3, 2025

(54) METHOD OF TREATING CHRONIC HEPATITIS B

(71) Applicant: GLAXOSMITHKLINE INTELLECTUAL PROPERTY DEVELOPMENT LIMITED, Stevenage (GB)

(72) Inventors: Ji Won CREMER, Apex, NC (US); Jessica Watson LIM, Los Angeles, CA (US); Mindy MAGEE, Phoenixville, PA (US); Ahmed NADER, Libertyville, IL (US); Geoffrey David QUINN, Hertfordshire (GB); Dickens THEODORE, Durham, NC (US); Amir YOUSSEF, East Brunswick, NJ (US)

(21) Appl. No.: 18/851,303

(22) PCT Filed: Mar. 27, 2023
(86) PCT No.: PCT/IB2023/053022

§ 371 Date: Sep. 26, 2024

Related U.S. Application Data
(60) Provisional application No. 63/324,662, filed on Mar. 29, 2022.
(60) Provisional application No. 63/365,535, filed on May 31, 2022.
(60) Provisional application No. 63/374,393, filed on Sep. 2, 2022.

(51) Int. Cl. C12N 15/113; A61K 9/00; A61K 45/06; A61P 31/20

(52) U.S. Cl. CPC C12N 15/1131; A61K 9/0019; A61K 45/06; A61P 31/20; C12N 2310/11

(57) ABSTRACT

The present disclosure is related to methods for treating chronic hepatitis B. The methods comprise administering to a subject in need thereof a therapeutically effective amount of bepirovirsen, wherein the subject has a HBsAg baseline level of not greater than a threshold level.

### FIELD

The present disclosure relates to methods for treating chronic hepatitis B. These methods comprise administering to a subject in need thereof a therapeutically effective amount of bepirovirsen, wherein the subject has a HBsAg baseline level of not greater than a threshold level.

### SEQUENCE LISTING

This application contains a Sequence Listing which has been submitted in XML format and is hereby incorporated by reference in its entirety. The XML copy is named 70153WO01_Seq_List.xml, is about 1,913 bytes in size and was created on Mar. 24, 2023.

### BACKGROUND

Hepatitis B virus (HBV) is a strict hepatotropic, partially double-stranded DNA virus. Although DNA is the genetic material, the replication cycle involves a reverse transcription step to copy a pregenomic RNA into DNA. Primary infection with HBV causes an acute hepatitis with symptoms of organ inflammation, fever, jaundice, and increased liver transaminases in blood. Those patients that are not able to overcome the acute virus infection suffer a chronic disease progression over many years with increased risk of developing cirrhotic liver or liver cancer.

HBV infection results in the production of two different particles: 1) the HBV virus itself (or Dane particle) which includes a viral capsid assembled from the HBV core antigen protein (HBcAg) and is covered by the hepatitis B surface antigen (HBsAg) and is capable of reinfecting cells and 2) subviral particles (or SVPs) which are high density lipoprotein-like particles comprised of lipids, cholesterol, cholesterol esters and the small and medium forms of the hepatitis B surface antigen (HBsAg) which are non-infectious. For each Dane particle produced, 1,000-10,000 SVPs are released into the blood. As such SVPs (and the HBsAg protein they carry) represent the overwhelming majority of viral protein in the blood. HBV infected cells also secrete a soluble proteolytic product of the pre-core protein called the HBV e-antigen (HBeAg). The presence of HBeAg in the serum of patients can serve as a marker of active replication in chronic hepatitis.

Currently the recommended therapies for chronic HBV infection by the American Association for the Study of Liver Diseases (AASLD) and the European Association for the Study of the Liver (EASL) include interferon alpha (IFN-α), pegylated interferon alpha-2a (Peg-IFN2α), entecavir, and tenofovir. However, typical interferon therapy is 48-weeks and is associated with tolerability issues due to side effects, and HBeAg seroconversion 24 weeks after therapy has ceased ranges from 27-36%. Seroconversion of HBsAg is even lower—approximately 3% observed immediately after treatment ceases, with an increase to upwards of 12% after 5 years.

The nucleoside and nucleotide therapies entecavir and tenofovir have been successful at reducing viral load (HBV DNA), but the rates of HBeAg seroconversion and HBsAg loss are even lower than those obtained using IFNα therapy. Other similar therapies, including lamivudine (3TC), telbivudine (LdT), and adefovir are also used, but for nucleoside/nucleotide therapies in general, the emergence of resistance limits therapeutic efficacy.

Thus, there is a need to discover and develop new anti-HBV therapies, more particularly, therapies capable of achieving functional cure for patients with chronic hepatitis B (CHB).

### SUMMARY

In one aspect, the present disclosure provides a method for treating a hepatitis B virus (HBV) infection in a subject in need thereof, the method comprising administering to the subject a therapeutically effective amount of bepirovirsen, wherein the subject has a HBsAg baseline level of not greater than a threshold level.

In one embodiment, the present disclosure provides a method for treating chronic hepatitis B (CHB) in a human in need thereof, the method comprising administering to the human a therapeutically effective amount of bepirovirsen, wherein the human has a HBsAg baseline level of not greater than a threshold level.

In another aspect, the present disclosure provides a method for treating chronic hepatitis B in a human in need thereof, the method comprising:
(a) determining that the human has a HBsAg baseline level of not greater than a threshold level; and
(b) administering to the human a therapeutically effective amount of bepirovirsen.

In another aspect, the present disclosure provides a method for treating chronic hepatitis B in a human in need thereof, the method comprising:
(a) measuring the HBsAg baseline level of the human;
(b) comparing the HBsAg baseline level with a threshold level; and
(c) if the HBsAg baseline level is not greater than the threshold level, then administering to the human a therapeutically effective amount of bepirovirsen.

In another aspect, the present disclosure provides a method for determining an increased likelihood of pharmacological effectiveness of treatment by bepirovirsen in a human with chronic hepatitis B, the method comprising:
(a) obtaining the HBsAg baseline level of the human; and
(b) comparing the HBsAg baseline level with a threshold level, wherein the HBsAg baseline level being not greater than the threshold level indicates an increased likelihood of pharmacological effectiveness of the treatment.

In another aspect, the present disclosure provides a method for increasing response rate for treating chronic hepatitis B by bepirovirsen in a human in need thereof, the method comprising:
(a) obtaining the HBsAg baseline level of the human;
(c) comparing the HBsAg baseline level with a threshold level; and
(b) if the HBsAg baseline level is not greater than a threshold level, then administering to the human a therapeutically effective amount of bepirovirsen.

In another aspect, the present disclosure provides bepirovirsen for use in a method of treating chronic hepatitis B in a human in need thereof, wherein the human has a HBsAg baseline level of not greater than a threshold level.

In another aspect, the present disclosure provides bepirovirsen for use in a method of treating chronic hepatitis B in a human in need thereof, the method comprising:
(a) determining that the human has a HBsAg baseline level of not greater than a threshold level; and
(b) administering to the human a therapeutically effective amount of bepirovirsen.

### BRIEF DESCRIPTION OF THE DRAWINGS

FIG. 1 depicts the study design of a Phase 2b clinical trial to assess efficacy and safety of treatment with bepirovirsen in patients with chronic hepatitis B (CHB).

FIG. 2 depicts the percentage of ON-NUC subjects with HBsAg<LLOQ, ≥LLOQ−<100 IU/mL, and ≥100 IU/mL over time by treatment arm.

FIG. 3 depicts the percentage of NOT ON-NUC subjects with HBsAg<LLOQ, ≥LLOQ−<100 IU/mL, and ≥100 IU/mL over time by treatment arm.

FIG. 4 depicts the proportion of ON-NUC subjects with virologic response in the end of treatment analysis window by subgroup based on HBeAg baseline levels by treatment arm.

FIG. 5 depicts the proportion of ON-NUC subjects with virologic response in the end of treatment analysis window by subgroup based on HBsAg baseline levels by treatment arm.

FIG. 6 depicts the proportion of NOT ON-NUC subjects with virologic response in the end of treatment analysis window by subgroup based on HBeAg baseline levels by treatment arm.

FIG. 7 depicts the proportion of NOT ON-NUC subjects with virologic response in the end of treatment analysis window by subgroup based on HBsAg baseline levels by treatment arm.

FIG. 8 depicts the proportion of NOT ON-NUC subjects with virologic response in the end of treatment analysis window by subgroup based on HBV DNA baseline levels by treatment arm.

FIGS. 9A and 9B depict observed and model-predicted individual subject PK, HBsAg, and ALT profiles of one subject whose HBsAg level increased after the end of bepirovirsen treatment.

FIGS. 10A and 10B depict observed and model-predicted individual subject PK, HBsAg, and ALT profiles of one subject whose HBsAg level remained low after the end of bepirovirsen treatment.

FIGS. 11A-11C depict the predicted percentages of subjects who achieved HBsAg<LLOQ at 12 weeks, at 24 weeks, and at 48 weeks (during the off-treatment period).

FIG. 12 depicts proportion of ON-NUC subjects achieving HBsAg<LLOQ and HBV DNA<LLOQ over time by treatment arm.

FIG. 13 depicts proportion of NOT ON-NUC subjects achieving HBsAg<LLOQ and HBV DNA<LLOQ over time by treatment arm.

FIG. 14 depicts the study design of a Phase 3 clinical trial to assess efficacy and safety of treatment with bepirovirsen in patients with chronic hepatitis B (CHB).

### DETAILED DESCRIPTION

### Definitions

Unless defined otherwise, all technical and scientific terms used herein have the same meanings as commonly understood by one of ordinary skill in the art. For example, certain terms used herein are defined as described in “A multilingual glossary of biotechnological terms: (IUPAC Recommendations),” Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise,” and variations such as “comprises” and “comprising,” will be understood to imply the inclusion of a stated element, integer, or step, or group of elements, integers, or steps but not the exclusion of any other element, integer, or step, or group of elements, integers, or steps.

Unless otherwise indicated, the following terms have the following meanings:

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass±10% of the recited value and the range is included.

“Antisense oligonucleotide (ASO)” refers to a single-stranded oligonucleotide having a nucleobase sequence that permits hybridization to a corresponding region or segment of a target nucleic acid.

“Baseline” values of certain substances (e.g., HBsAg, HBeAg, HBsAb, HBeAb, HBV DNA, ALT) are measured in the blood samples taken from patients before the first dose of treatment.

Bepirovirsen is an ASO currently in clinical evaluation for treating HBV infections. It is compound ISIS No. 505358 as disclosed in WO2012/145697. Bepirovirsen has 20 linked nucleosides and has a nucleobase sequence of 5′-GCAGAGGTGAAGCGAAGTGC-3′ (SEQ ID NO:1), and it includes:
a gap segment consisting of ten linked deoxynucleosides,
a 5′ wing segment consisting of 5 linked nucleosides, and
a 3′ wing segment consisting of 5 linked nucleosides,
wherein the gap segment is positioned between the 5′ wing segment and the 3′ wing segment, wherein each nucleoside of each wing segment includes a 2′-O-methoxyethyl sugar, wherein each intemucleoside linkage is a phosphorothioate linkage, and wherein each cytosine is a 5-methylcytosine. The CAS Registry Number of bepirovirsen is 1403787-62-1.

“Chronic hepatitis B (CHB) infection” occurs when a person initially suffers from an acute infection but is then unable to fight off the infection. About 90% of infants infected at birth will progress to chronic disease. However, as a person ages, the risk of chronic infection decreases such that between 20%-50^{%} of people infected as children and less than 10% of people infected as adults will progress from acute to chronic infection. The terms “chronic hepatitis B infection”, “chronic hepatitis B”, “chronic HBV infection”, and “CHB” are used interchangeably herein.

“Dose” refers to a specified quantity of an active pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

“Dosing regimen” is a combination of doses designed to achieve one or more desired effects.

“Duration” refers to the period of time during which an activity or event continues. In certain embodiments, the duration of treatment is the period of time during which doses of a pharmaceutical agent are administered.

“Functional cure” refers to sustained suppression (24 weeks or longer) of HBV DNA (<LLOQ) off all HBV treatment with HBsAg loss (<0.05 IU/mL) with or without HBsAb after a finite duration of therapy.

“HBV” refers to mammalian hepatitis B virus, including human hepatitis B virus. The term encompasses geographical genotypes of hepatitis B virus, particularly human hepatitis B virus, as well as variant strains of geographical genotypes of hepatitis B virus. Human geographical genotypes of HBV include genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); and H (Central America).

“HBV antigen” refers to any hepatitis B virus antigen or protein, including core proteins such as “hepatitis B core antigen” or “HBcAg,” “hepatitis B E antigen” or “HBeAg,” and envelope proteins such as “HBV surface antigen” or “HBsAg.”

“Hepatitis B E antigen” or “HBeAg” is a secreted, non-particulate form of HBV core protein. HBV antigens HBeAg and HBcAg share primary amino acid sequences, so show cross-reactivity at the T cell level. HBeAg is not required for viral assembly or replication, although studies suggest they may be required for establishment of chronic infection.

“HBV surface antigen,” “HBsAg,” or “HBs” is the envelope protein of infectious HBV viral particles (Dane Particles), but is also secreted as a non-infectious particle (subviral particle, SVP) with serum levels 1000-fold higher than HBV viral particles. The serum levels of HBsAg in an infected person or animal can be as high as 1000 μg/mL (Kann and Gehrlich (1998) Topley & Wilson's Microbiology and Microbial Infections, 9th ed. 745).

“HBV-related condition” refers to any disease, biological condition, medical condition, or event which is exacerbated, caused by, related to, associated with, or traceable to a hepatitis B virus infection, exposure, or illness. The term hepatitis B-related condition includes chronic HBV infection, inflammation, fibrosis, cirrhosis, liver cancer, serum hepatitis, jaundice, liver inflammation, liver fibrosis, liver cirrhosis, liver failure, diffuse hepatocellular inflammatory disease, hemophagocytic syndrome, HBV viremia, liver disease related to transplantation, and conditions having symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen.

“Induce,” “inhibit,” “potentiate,” “elevate,” “increase,” “decrease” or the like, generally denote quantitative differences between two states. Such terms may refer to a statistically significant difference between the two states. For example, “an amount effective to inhibit the activity or expression of HBV” refers to the level of activity or expression of HBV in a treated cell and this will quantitatively differ, and may be statistically significant, from the level of HBV activity or expression in untreated cells. Such terms are applied to, for example, levels of expression and levels of activity.

“Nucleobase” refers to a heterocyclic moiety capable of pairing with a base of another nucleic acid.

“Nucleobase sequence” refers to the order of contiguous nucleobases independent of any sugar, linkage, and/or nucleobase modification.

“Pharmaceutically acceptable salts” refer to physiologically acceptable salts of compounds, i.e., salts that retain the desired biological activity of the parent active ingredients and do not impart undesired toxicological effects thereto.

“Phosphorothioate linkage” refers to a linkage between nucleotides where the phosphodiester bond is modified by replacing one of the non-bridging oxygen atoms with a sulfur atom. A phosphorothioate linkage is a modified internucleoside linkage.

“Region” is defined as a portion of, for example, a nucleic acid having at least one identifiable structure, function, or characteristic.

“Stable nucleoside or nucleotide analogue (NA) therapy” is defined as no changes to the nucleoside or nucleotide analogue regimen for at least 6 months prior to the treatment and with no planned changes to the regimen for the duration of the treatment.

“Segments” may refer to smaller, or sub-portions of, regions within, for example, a nucleic acid.

“Seroclearance” refers to the clearance or removal of an antigen (like HBsAg and/or HBV DNA levels) from the blood (or a measurement below the lower limit of quantification (i.e. <LLOQ) by the lab) in a CHB patient following treatment. In testing human samples, when serum HBsAg level is measured by a sandwich immunoassay with COBAS HBsAg quant II (Roche), the LLOQ is 0.05 IU/mL. In testing human samples, when the serum HBV DNA level is measured with COBAS Ampliprep/COBAS Tagman HBV test v.2.0 (Roche), the LLOQ is 20 IU/mL.

“Subject” refers to a human or non-human animal selected for treatment or therapy. In one embodiment, the subject is a human.

“Therapeutically effective amount” refers to the administration of a pharmaceutical agent to a subject, either alone or as part of a pharmaceutical composition and either in a single dose or as part of a series of doses, in an amount capable of having any detectable, positive effect on any symptom, aspect, or characteristic of a disease or condition when administered to the subject.

“Treatment” refers to administering a pharmaceutical agent to a subject to affect an alteration or improvement of the disease or condition. The term “treating” as used herein in relation to chronic hepatitis B infection refers to the administration of suitable compositions with the intention of reducing the symptoms of CHB, preventing the progression of CHB or reducing the level of one or more detectable markers of CHB.

### Methods

The present disclosure provides a method for treating a hepatitis B virus (HBV) infection in a subject in need thereof, the method comprising administering to the subject a therapeutically effective amount of bepirovirsen, wherein the subject has a HBsAg baseline level of not greater than a threshold level. In some embodiments, the HBV infection is chronic hepatitis B (CHB).

In another aspect, the present disclosure provides a method for treating chronic hepatitis B in a human in need thereof, the method comprising:
(a) determining that the human has a HBsAg baseline level of not greater than a threshold level; and
(b) administering to the human a therapeutically effective amount of bepirovirsen.

The HBsAg baseline level in the blood of a CHB patient is measured by methods known in the art before the start of treatment. As used herein, the step “determining” can be achieved by measuring the HBsAg baseline level of the patient and comparing the baseline level with a predetermined threshold level. The step “determining” can also be achieved by receiving the HBsAg baseline level information from the patient or a testing facility and comparing the HBsAg baseline level with a predetermined threshold level.

In another aspect, the present disclosure provides a method for treating chronic hepatitis B in a human in need thereof, the method comprising:
(a) measuring the HBsAg baseline level of the human;
(b) comparing the HBsAg baseline level with a threshold level; and
(c) if the HBsAg baseline level is not greater than the threshold level, then administering to the human a therapeutically effective amount of bepirovirsen.

In another aspect, the present disclosure provides a method for determining an increased likelihood of pharmacological effectiveness of treatment by bepirovirsen in a human with chronic hepatitis B, the method comprising:
(a) obtaining the HBsAg baseline level of the human; and
(b) comparing the HBsAg baseline level with a threshold level,
wherein the HBsAg baseline level being not greater than the threshold level indicates an increased likelihood of pharmacological effectiveness of the treatment.

As used herein, the step “obtaining” can be achieved by measuring the HBsAg baseline level of the patient directly or receiving the HBsAg baseline level information from the patient or a testing facility.

In another aspect, the present disclosure provides a method for increasing response rate for treating chronic hepatitis B by bepirovirsen in a human in need thereof, the method comprising:
(a) obtaining the HBsAg baseline level of the human;
(c) comparing the HBsAg baseline level with a threshold level; and
(b) if the HBsAg baseline level is not greater than a threshold level, then administering to the human a therapeutically effective amount of bepirovirsen.

It was reported that a low HBsAg baseline level can be used as a predictor of HBsAg loss in HBeAg-negative patients treated with PEG-IFN and adefovir. See Takkenberg, et al., “Baseline hepatitis B surface antigen (HBsAg) as predictor of sustained HBsAg loss in chronic hepatitis B patients treated with pegylated interferon-α2a and adefovir,” Antivir. Ther. 2013; 18(7):895-904. In particular, a HBsAg baseline level below 400 IU/mL was identified as a good predictor for treating HBeAg-negative patients with PEG-IFN and adefovir. However, a low HBsAg baseline level could not predict HBsAg loss in HBeAg-positive patients in the same study.

In the methods as disclosed herein, bepirovirsen can be administered as a free acid, a pharmaceutically acceptable salt thereof (e.g., a sodium salt), or a combination thereof. In some embodiments, bepirovirsen is administered as a free acid. In some embodiments, bepirovirsen is administered as a pharmaceutically acceptable salt thereof (e.g., a sodium salt). In some embodiments, bepirovirsen is administered as a combination of a free acid and a sodium salt. In some embodiments, bepirovirsen is administered by subcutaneous injection.

As used herein, the “therapeutically effective amount of bepirovirsen” refers to the amount of bepirovirsen free acid. In some embodiments, the therapeutically effective amount of bepirovirsen is about 150 mg to 450 mg once weekly. In some embodiments, the therapeutically effective amount of bepirovirsen is about 150 mg, 200 mg, 250 mg, 300 mg, 350 mg, 400 mg, or 450 mg once weekly, or in a range between any two preceding values. In some embodiments, the therapeutically effective amount of bepirovirsen is about 150 mg once weekly. In some embodiments, the therapeutically effective amount of bepirovirsen is about 300 mg once weekly. In some embodiments, bepirovirsen is administered weekly with additional loading doses in the first two weeks on Day 4 and Day 11 following the first dose. In some embodiments, bepirovirsen is administered at a dose of about 300 mg once weekly with additional loading doses in the first two weeks on Day 4 and Day 11 following the first dose. In a particular embodiment, the loading dose is 300 mg.

In some embodiments, bepirovirsen is administered for about 12 to 48 weeks. In some embodiments, bepirovirsen is administered for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, or 48 weeks, or for a range between any two preceding periods. In one embodiment, bepirovirsen is administered for 12 weeks. In one embodiment, bepirovirsen is administered for 24 weeks. In one embodiment, bepirovirsen is administered for 36 weeks. In one embodiment, bepirovirsen is administered for 48 weeks. In one embodiment, bepirovirsen and is administered for 12 weeks or 24 weeks, with additional loading doses on Day 4 and Day 11 following the first dose.

In some embodiments, bepirovirsen is administered at a dose of about 300 mg once weekly for 24 weeks. In some embodiments, bepirovirsen is administered at a dose of about 300 mg once weekly for 12 weeks. In some embodiments, bepirovirsen is administered at a dose of about 300 mg once weekly for 12 weeks, and then at a dose of about 150 mg once weekly for 12 weeks.

In some embodiments, bepirovirsen is administered at a dose of about 300 mg once weekly for 24 weeks, with additional loading doses each of 300 mg, on Day 4 and Day 11 following the first dose. In some embodiments, bepirovirsen is administered at a dose of about 300 mg once weekly for 12 weeks, with additional loading doses each of 300 mg on Day 4 and Day 11 following the first dose.

In some embodiments, the present disclosure provides a method for treating CHB in a human in need thereof comprising administering to the human a therapeutically effective amount of bepirovirsen, wherein the subject has a HBsAg baseline level of not greater than a threshold level. In some embodiments, the threshold level of the HBsAg baseline is in the range of about 500 IU/mL to 10,000 IU/mL, i.e., about 2.7 log 10 IU/mL to 4.0 log 10 IU/mL.

In some embodiments, the threshold level of the HBsAg baseline is about 500, 600, 700, 800, 900, 1000, 1100, 1200, 1300, 1400, 1500, 1600, 1700, 1800, 1900, 2000, 3000, 4000, 5000, 6000, 7000, 8000, 9000, or 10,000 IU/mL, or in a range between any two of the preceding numbers. In some embodiments, the threshold level of the HBsAg baseline is about 1000, 2000, or 3000 IU/mL. In some embodiments, the threshold level of the HBsAg baseline is about 1000 IU/mL. In some embodiments, the threshold level of the HBsAg baseline is about 3000 IU/mL. In some embodiments, the threshold level of the HBsAg baseline is about 2.7, 2.8, 2.9, 3.0, 3.1, 3.2, 3.3, 3.4, 3.5, 3.6, 3.7, 3.8, 3.9, or 4.0 log 10 IU/mL, or in a range between any two of the preceding numbers. In some embodiments, the threshold level of the HBsAg baseline is about 3.0 to 4.0 log 10 IU/mL. In some embodiments, the threshold level of the HBsAg baseline is about 3.0 to 3.5 log 10 IU/mL. In some embodiments, the threshold level of the HBsAg baseline is about 3.0 log 10 IU/mL. In some embodiments, the threshold level of the HBsAg baseline is about 3.5 log 10 IU/mL.

In one embodiment, the present disclosure provides a method for treating CHB in a human in need thereof, the method comprising administering to the human a therapeutically effective amount of bepirovirsen, wherein the human has a HBsAg baseline level of not greater than about 1000 IU/mL. In one embodiment, the present disclosure provides a method for treating CHB in a human in need thereof, the method comprising administering to the human a therapeutically effective amount of bepirovirsen, wherein the human has a HBsAg baseline level of not greater than about 3000 IU/mL.

In one embodiment, the present disclosure provides a method for treating CHB in a human in need thereof, the method comprising administering bepirovirsen to the human at a dose of 300 mg once weekly for 24 weeks, wherein the human has a HBsAg baseline level of not greater than about 1000 IU/mL. In one embodiment, the present disclosure provides a method for treating CHB in a human in need thereof, the method comprising administering bepirovirsen to the human at a dose of 300 mg once weekly for 24 weeks, wherein the human has a HBsAg baseline level of not greater than about 3000 IU/mL.

In one embodiment, the present disclosure provides a method for treating CHB in a human in need thereof, the method comprising administering bepirovirsen to the human at a dose of 300 mg once weekly for 24 weeks, wherein the human has a HBsAg baseline level of not greater than about 1000 IU/mL and is HBeAg negative. In one embodiment, the present disclosure provides a method for treating CHB in a human in need thereof, the method comprising administering bepirovirsen to the human at a dose of 300 mg once weekly for 24 weeks, wherein the human has a HBsAg baseline level of not greater than about 3000 IU/mL and is HBeAg negative.

In one embodiment, the present disclosure provides a method for treating CHB in a human in need thereof, the method comprising administering bepirovirsen to the human at a dose of 300 mg once weekly for 24 weeks, wherein the human has a HBsAg baseline level of not greater than about 1000 IU/mL and is HBeAg negative, and wherein the human is on stable nucleoside or nucleotide analogue (NA) therapy. In one embodiment, the present disclosure provides a method for treating CHB in a human in need thereof, the method comprising administering bepirovirsen to the human at a dose of 300 mg once weekly for 24 weeks, wherein the human has a HBsAg baseline level of not greater than about 3000 IU/mL and is HBeAg negative, and wherein the human is on stable nucleoside or nucleotide analogue (NA) therapy.

In some embodiments, the human is not treated with another HBsAg reducing agent or immunomodulating agent. That is, bepirovirsen is used as monotherapy for treating CHB. A HBsAg reducing agent can be a small/short interfering RNA (siRNA), an antisense oligonucleotide (ASO), a nucleic acid polymer (NAP), an HBV RNA destabilizer, an HBV specific neutralizing mAb, or a combination thereof. An immunomodulating agent can be IFN-α, PEG-IFN-α, a therapeutic vaccine, a PD-1/PD-L1 inhibitor, a TLR7 agonist, a TLR8 agonist, a TLR9 agonist, or a combination thereof.

In one embodiment, the human is on stable nucleoside or nucleotide analogue (NA) therapy (e.g., tenofovir disoproxil, tenofovir alafenamide, or entecavir). In some embodiments, the NA therapy is lamivudine, adefovir, adefovir dipivoxil, telbivudine, entecavir, tenofovir, tenofovir disoproxil fumarate (TDF), or tenofovir alafenamide (TAF), or a pharmaceutically acceptable salt thereof. In some embodiments, the NA therapy is entecavir, tenofovir, tenofovir disoproxil fumarate, or tenofovir alafenamide. In some embodiments, the NA therapy is entecavir. In some embodiments, the NA therapy is tenofovir. In some embodiments, the NA therapy is tenofovir disoproxil fumarate. In some embodiments, the NA therapy is tenofovir alafenamide.

In another embodiment, the subject is not on NA therapy. In some embodiments, the subject is treatment-naïve.

In some embodiments, the human has a higher chance of achieving HBsAg below LLOQ at the end of the bepirovirsen treatment as compared to those with a HBsAg baseline level of greater than the threshold level. In some embodiments, the human has at least 5%, 10%, 15%, 20%, 25%, or 30% higher chance of achieving HBsAg below LLOQ at the end of the bepirovirsen treatment. In some embodiment, the human has at least 5%, 10%, 15%, 20%, 25%, or 30% higher chance of achieving HBsAg below LLOQ 24 weeks after the end of the bepirovirsen treatment.

In some embodiments, the human has a higher chance of achieving HBV DNA below LLOQ at the end of the bepirovirsen treatment as compared to those with a HBsAg baseline level of greater than the threshold level. In some embodiments, the human has at least 5%, 10%, 15%, 20%, 25%, or 30% higher chance of achieving HBV DNA below LLOQ at the end of the bepirovirsen treatment. In some embodiment, the human has at least 5%, 10%, 15%, 20%, 25%, or 30% higher chance of achieving HBV DNA below LLOQ 24 weeks after the end of the bepirovirsen treatment.

In some embodiments, the human with a HBsAg baseline level of not greater than a threshold level has a higher chance of achieving HBsAg and HBV DNA below LLOQ at the end of the bepirovirsen treatment as compared to those with a HBsAg baseline level of greater than the threshold level. In some embodiments, the human with a HBsAg baseline level of not greater than a threshold level has at least 5%, 10%, 15%, 20%, 25%, or 30% higher chance of achieving HBsAg and HBV DNA below LLOQ at the end of the bepirovirsen treatment. In some embodiments, the human with a HBsAg baseline level of not greater than a threshold level has at least 5%, 10%, 15%, 20%, 25%, or 30% higher chance of achieving HBsAg and HBV DNA below LLOQ 24 weeks after the end of the bepirovirsen treatment.

In some embodiments, the hepatitis B virus infection is caused by any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America). In some embodiments, the subject has chronic hepatitis B (CHB).

In some embodiments, the subject is HBeAg negative or HBeAg positive prior to treatment. In some embodiments, the subject is HBeAg negative prior to treatment. In some embodiments, the subject is HBeAg positive prior to treatment.

In another aspect, the present disclosure provides bepirovirsen for use in a method of treating chronic hepatitis B in a human in need thereof, wherein the human has a HBsAg baseline level of not greater than a threshold level.

In another aspect, the present disclosure provides bepirovirsen for use in a method of treating chronic hepatitis B in a human in need thereof, the method comprising:
(a) determining that the human has a HBsAg baseline level of not greater than a threshold level; and
(b) administering to the human a therapeutically effective amount of bepirovirsen.

### Compositions

The present disclosure provides a composition comprising bepirovirsen for treating a hepatitis B virus (HBV) infection in a subject in need thereof, the method of treatment comprising administering to the subject a therapeutically effective amount of bepirovirsen, wherein the subject has a HBsAg baseline level of not greater than a threshold level. In some embodiments, the HBV infection is chronic hepatitis B (CHB).

The present disclosure also provides bepirovirsen for treating a hepatitis B virus (HBV) infection in a subject in need thereof, the method comprising administering to the subject a therapeutically effective amount of bepirovirsen, wherein the subject has a HBsAg baseline level of not greater than a threshold level. In some embodiments, the HBV infection is chronic hepatitis B (CHB).

The present invention also provides a composition comprising bepirovirsen, or bepirovirsen, for use in a method of treating chronic hepatitis B in a human in need thereof, wherein the method comprises: (a) determining that the human has a HBsAg baseline level of not greater than a threshold level; and (b) administering to the human a therapeutically effective amount of bepirovirsen.

In other embodiments of the composition or bepirovirsen, the method of administering the composition or bepirovirsen can be according to any of the methods detailed above.

For example, in some embodiments, the threshold level of the HBsAg baseline is about 500, 600, 700, 800, 900, 1000, 1100, 1200, 1300, 1400, 1500, 1600, 1700, 1800, 1900, 2000, 3000, 4000, 5000, 6000, 7000, 8000, 9000, or 10,000 IU/mL, or in a range between any two of the preceding numbers. In some embodiments, the threshold level of the HBsAg baseline is about 1000, 2000, or 3000 IU/mL. In some embodiments, the threshold level of the HBsAg baseline is about 1000 IU/mL. In some embodiments, the threshold level of the HBsAg baseline is about 3000 IU/mL. In some embodiments, the threshold level of the HBsAg baseline is about 2.7, 2.8, 2.9, 3.0, 3.1, 3.2, 3.3, 3.4, 3.5, 3.6, 3.7, 3.8, 3.9, or 4.0 log 10 IU/mL, or in a range between any two of the preceding numbers. In some embodiments, the threshold level of the HBsAg baseline is about 3.0 to 4.0 log 10 IU/mL. In some embodiments, the threshold level of the HBsAg baseline is about 3.0 to 3.5 log 10 IU/mL. In some embodiments, the threshold level of the HBsAg baseline is about 3.0 log 10 IU/mL. In some embodiments, the threshold level of the HBsAg baseline is about 3.5 log 10 IU/mL.

In one embodiment, the present disclosure provides bepirovirsen for use in a method of treating CHB in a human in need thereof, wherein the method comprises administering to the human a therapeutically effective amount of bepirovirsen, wherein the human has a HBsAg baseline level of not greater than about 1000 IU/mL. In one embodiment, the present disclosure provides bepirovirsen for use in a method of treating CHB in a human in need thereof, the method comprising administering to the human a therapeutically effective amount of bepirovirsen, wherein the human has a HBsAg baseline level of not greater than about 3000 IU/mL.

In another embodiment, the present disclosure provides bepirovirsen for use in a method of treating CHB in a human in need thereof, wherein the method comprises administering bepirovirsen to the human at a dose of 300 mg once weekly for 24 weeks, wherein the human has a HBsAg baseline level of not greater than about 1000 IU/mL. In one embodiment, the present disclosure provides bepirovirsen for use in a method of treating CHB in a human in need thereof, the method comprising administering bepirovirsen to the human at a dose of 300 mg once weekly for 24 weeks, wherein the human has a HBsAg baseline level of not greater than about 3000 IU/mL.

In another embodiment, the present disclosure provides bepirovirsen for use in a method of treating CHB in a human in need thereof, wherein the method comprises administering bepirovirsen to the human at a dose of 300 mg once weekly for 12 weeks, wherein the human has a HBsAg baseline level of not greater than about 1000 IU/mL. In one embodiment, the present disclosure provides bepirovirsen for use in a method of treating CHB in a human in need thereof, the method comprising administering bepirovirsen to the human at a dose of 300 mg once weekly for 12 weeks, wherein the human has a HBsAg baseline level of not greater than about 3000 IU/mL.

### EMBODIMENTS

The present disclosure is also illustrated by the following Embodiments:

1. A method for treating chronic hepatitis B in a human in need thereof, the method comprising administering to the human a therapeutically effective amount of bepirovirsen, wherein the human has a HBsAg baseline level of not greater than a threshold level.

2. A method for treating chronic hepatitis B in a human in need thereof, the method comprising:
(a) determining that the human has a HBsAg baseline level of not greater than a threshold level; and
(b) administering to the human a therapeutically effective amount of bepirovirsen.

3. A method for treating chronic hepatitis B in a human in need thereof, the method comprising:
(a) measuring the HBsAg baseline level of the human;
(b) comparing the HBsAg baseline level with a threshold level; and
(c) if the HBsAg baseline level is not greater than the threshold level, then administering to the human a therapeutically effective amount of bepirovirsen.

4. A method for determining an increased likelihood of pharmacological effectiveness of treatment by bepirovirsen in a human with chronic hepatitis B, the method comprising
(a) obtaining the HBsAg baseline level of the human; and
(b) comparing the HBsAg baseline level with a threshold level, wherein the HBsAg baseline level being not greater than the threshold level indicates an increased likelihood of pharmacological effectiveness of the treatment.

5. A method for increasing response rate for treating chronic hepatitis B by bepirovirsen in a human in need thereof, the method comprising:
(a) obtaining the HBsAg baseline level of the human;
(b) comparing the HBsAg baseline level with a threshold level; and
(c) if the HBsAg baseline level is not greater than the threshold level, then administering to the human a therapeutically effective amount of bepirovirsen.

6. The method of any one of Embodiments 1 to 5, wherein the threshold level is in the range of about 500 IU/mL to 4000 IU/mL.

7. The method of any one of Embodiments 1 to 5, wherein the threshold level is about 1000 IU/mL.

8. The method of any one of Embodiments 1 to 5, wherein the threshold level is about 3000 IU/mL.

9. The method of any one of Embodiments 1 to 8, wherein the human is not treated with another HBsAg reducing agent or immunomodulating agent.

10. The method of Embodiment 9, wherein the HBsAg reducing agent is an siRNA, an antisense oligonucleotide, a nucleic acid polymer (NAP), an HBV RNA destabilizer, an HBV specific neutralizing mAb, or a combination thereof.

11. The method of Embodiment 9, wherein the immunomodulating agent is IFN-α, PEG-IFN-α, a therapeutic vaccine, a PD-1/PD-L1 inhibitor, a TLR7 agonist, a TLR8 agonist, a TLR9 agonist, or a combination thereof.

12. The method of any one of Embodiments 1 to 11, wherein bepirovirsen is administered by subcutaneous injection.

13. The method of any one of Embodiments 1 to 12, wherein bepirovirsen is administered at a dose of about 150 mg to 450 mg once weekly.

14. The method of any one of Embodiments 1 to 12, wherein bepirovirsen is administered at a dose of about 150 mg or 300 mg once weekly.

15. The method of any one of Embodiments 1 to 12, wherein bepirovirsen is administered at a dose of about 300 mg once weekly.

16. The method of any one of Embodiments 1 to 15, wherein bepirovirsen is administered for 12 weeks to 48 weeks.

17. The method of any one of Embodiments 1 to 15, wherein bepirovirsen is administered for 12 weeks or 24 weeks.

18. The method of any one of Embodiments 1 to 15, wherein bepirovirsen is administered for 12 weeks.

19. The method of any one of Embodiments 1 to 15, wherein bepirovirsen is administered for 24 weeks.

20. The method of any one of Embodiments 1 to 12, wherein bepirovirsen is administered at a dose of about 300 mg once weekly for 24 weeks.

21. The method of any one of Embodiments 1 to 12, wherein bepirovirsen is administered at a dose of about 300 mg once weekly for 12 weeks.

22. The method of any one of Embodiments 1 to 12, wherein bepirovirsen is administered at a dose of about 300 mg once weekly for 12 weeks, and then at a dose of about 150 mg once weekly for 12 weeks.

23. The method of any one of Embodiments 13 to 22, wherein bepirovirsen is administered weekly with additional loading doses in the first two weeks on Day 4 and Day 11 following the first dose.

24. The method of embodiment 23, wherein the loading dose is 300 mg.

25. The method of any one of Embodiments 1 to 24, wherein the human is on stable nucleoside or nucleotide analogue (NA) therapy.

26. The method of Embodiment 25, wherein the NA therapy is lamivudine, adefovir, adefovir dipivoxil, telbivudine, entecavir, tenofovir, tenofovir disoproxil fumarate, or tenofovir alafenamide, or a pharmaceutically acceptable salt thereof.

27. The method of Embodiments 25 or 26, wherein the human discontinues the NA therapy 24 weeks after the end of the bepirovirsen treatment.

28. The method of any one of Embodiments 1 to 24, wherein the human is not treated with a nucleoside or nucleotide analogue (NA).

29. The method of any one of Embodiments 1 to 28, wherein the human is HBeAg negative or HBeAg positive prior to treatment.

30. The method of Embodiment 29, wherein the human is HBeAg negative prior to treatment.

31. The method of Embodiment 29, wherein the human is HBeAg positive prior to treatment.

32. The method of any one of Embodiments 1 to 31, wherein the human has a higher chance of achieving HBsAg below LLOQ at the end of the bepirovirsen treatment as compared to those with a HBsAg baseline level of greater than the threshold level.

33. The method of Embodiment 32, wherein the human has at least 5%, 10%, 15%, 20%, 25%, or 30% higher chance of achieving HBsAg below LLOQ at the end of the bepirovirsen treatment.

34. The method of any one of Embodiments 1 to 33, wherein the human has a higher chance of achieving HBV DNA below LLOQ at the end of the bepirovirsen treatment as compared to those with a HBsAg baseline level of greater than the threshold level.

35. The method of Embodiment 34, wherein the human has at least 5%, 10%, 15%, 20%, 25%, or 30% higher chance of achieving HBV DNA below LLOQ at the end of the bepirovirsen treatment.

36. The method of any one of Embodiments 1 to 31, wherein the human has a higher chance of achieving HBsAg below LLOQ 24 weeks after the end of the bepirovirsen treatment as compared to those with a HBsAg baseline level of greater than the threshold level.

37. The method of Embodiment 36, wherein the human has at least 5%, 10%, 15%, 20%, 25%, or 30% higher chance of achieving HBsAg below LLOQ 24 weeks after the end of the bepirovirsen treatment.

38. The method of Embodiment 27, wherein the human has a higher chance of achieving HBsAg below LLOQ 24 weeks after the NA therapy discontinuation as compared to those with a HBsAg baseline level of greater than the threshold level.

39. The method of Embodiment 38, wherein the human has at least 5%, 10%, 15%, 20%, 25%, or 30% higher chance of achieving HBsAg below LLOQ 24 weeks after the NA therapy discontinuation.

40. Bepirovirsen for use in a method of any of the preceding embodiments.

41. Bepirovirsen for use in a method of treating chronic hepatitis B in a human in need thereof, wherein the human has a HBsAg baseline level of not greater than a threshold level.

42. Bepirovirsen for use in a method of treating chronic hepatitis B in a human in need thereof, the method comprising:
(a) determining that the human has a HBsAg baseline level of not greater than a threshold level; and
(b) administering to the human a therapeutically effective amount of bepirovirsen.

43. Use of bepirovirsen in the manufacture of a medicament for the treatment of chronic hepatitis B in a human in need thereof, wherein the human to be treated has a HBsAg baseline level of not greater than a threshold level.

### EXAMPLES

While aspects of the disclosure presented herein have been described more particularly in accordance with some embodiments, the following examples, which highlight certain features and properties of the exemplary embodiments of the disclosure described herein, serve only to illustrate the disclosure described herein and are not intended to limit the same.

### Example 1

### Evaluation of the Efficacy and Safety of Treatment with Bepirovirsen in Participants with Chronic Hepatitis B

### A. Study Design

This is a Phase 2b, multi-center, randomized, partial-blind [Sponsor and Participant blinded] study to assess efficacy and safety of treatment with bepirovirsen in two populations of patients with chronic hepatitis B (CHB): (1) participants on stable nucleos(t)ide treatment (ON-NUC) and (2) participants not currently on nucleos(t)ide therapy (NOT ON-NUC).

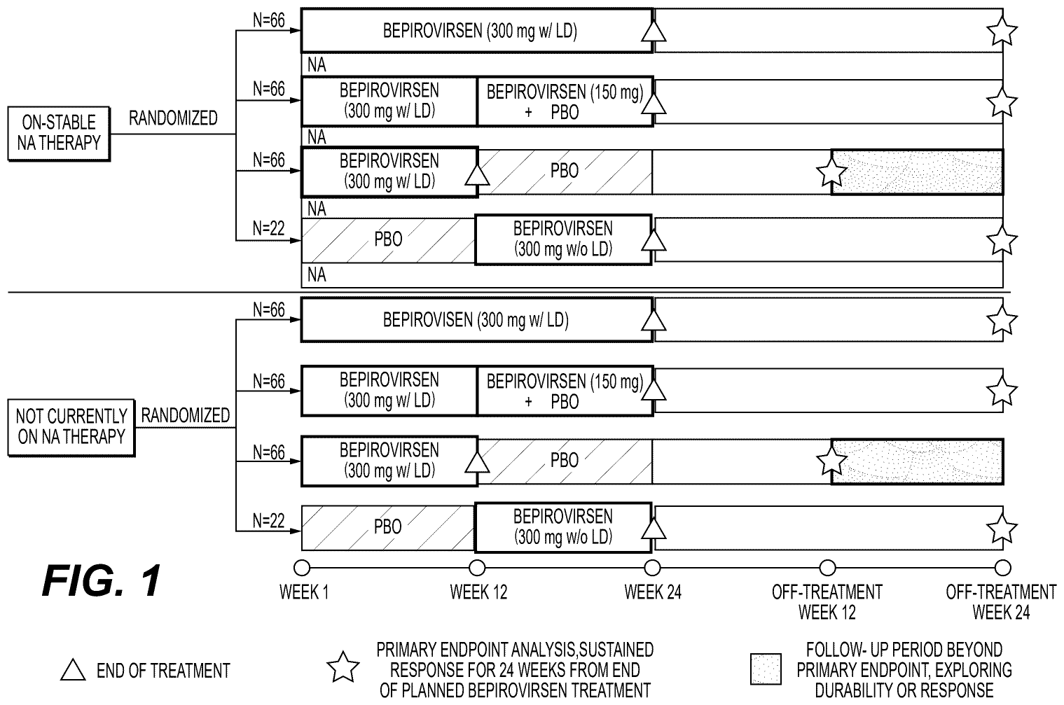

Both populations of patients with CHB were randomized into one of 4 parallel arms (see FIG. 1):
1. 300 mg bepirovirsen once/week for 24 weeks (plus loading dose of 300 mg bepirovirsen on Day 4 and Day 11);
2. 300 mg bepirovirsen once/week for 12 weeks (plus loading dose of 300 mg bepirovirsen on Day 4 and Day 11) followed by step-down in dose of 150 mg (plus placebo to match to maintain participant blinding) bepirovirsen once/week for 12 weeks;
3. 300 mg bepirovirsen once/week for 12 weeks (plus loading dose of 300 mg bepirovirsen on Day 4 and Day 11) followed by placebo once/week for 12 weeks;
4. Placebo once/week for 12 weeks followed by 300 mg bepirovirsen once/week for 12 weeks (plus placebo loading doses to match on Day 4 and Day 11, no loading dose for bepirovirsen treatment).

After the 24 weeks of dosing with bepirovirsen or placebo, there was a post-treatment follow-up period of 24 weeks.

Populations were stratified based on HBsAg level (HBsAg 3 log 10 IU/mL and >3 log 10 IU/mL) and whether participants were HBeAg positive or negative.

### B. Objectives and Endpoints

The primary objectives of this study are:

Efficacy: To assess the efficacy of the three dosing regimens of bepirovirsen in participants with CHB.

Primary estimands supporting the primary objective are defined as:
Population: separate assessment for the following:
   participants with CHB on stable nucleos(t)ide therapy
   participants with CHB not currently on nucleos(t)ide therapy
Variable: Participants achieving sustained virologic response (SVR) for 24 weeks after the planned end of bepirovirsen treatment in the absence of rescue medication. Sustained virologic response is defined as observing HBsAg<LLOQ and HBV DNA<LLOQ during the 24 weeks after the end of bepirovirsen treatment.
Treatments: arms 1, 2, and 3. Estimation of the within-arm response rate.
Intercurrent events: use of rescue medication, and discontinuation of/interruption of/adherence to Investigational Product (IP). The use of rescue medication was incorporated into the definition of variable (composite strategy). Discontinuation of, interruption of, and adherence to IP were ignored (treatment policy). Wide disruptive events (such as COVID-19 pandemic) leading to discontinuation of, interruption in, and non-adherance to bepirovirsen were handled assuming they had not happened (hypothetical strategy).
Population summary: proportion of participants who achieve SVR for each treatment arm.

The primary estimands for each sub-population is the proportion of participants in each treatment arm 1, 2, and 3 who achieve SVR for 24 weeks after the planned end of bepirovirsen treatment in the absence of rescue medication regardless of completing IP, interruptions in IP or adherence to IP had they not been affected by wide disruptive events.

A supplementary estimand is defined to support the primary objective:
the supplementary estimand is defined in the same way as the main estimand, except the assessment time frame for participants achieving SVR is 24 weeks after the actual end of treatment. Therefore, the strategy for intercurrent events of treatment discontinuation is while-on-treatment. This supplementary estimand supporting the primary objective in participants with CHB on stable nucloes(t)ide therapy and participants with CHB not currently on nucleos(t)ide therapy is the proportion of participants in each treatment arm 1, 2, and 3 who achieve SVR for 24 weeks after the actual end of bepirovirsen treatment in the absence of rescue medication, regardless of completing IP, interruptions in IP or adherence to IP, had they not been affected by wide disruptive events.

The secondary objectives of this study are:
1. Efficacy: To assess the efficacy of bepirovirsen on biomarkers and virus-specific antibody responses;
2. Efficacy: To compare the efficacy between 12 weeks, 12 weeks+12 weeks step-down, and 24 weeks of bepirovirsen treatment;
3. Pharmacokinetics (PK): To characterize bepirovirsen and nucleos(t)ide PK in participants with CHB.
   The safety objectives of this study are:

To assess the safety and tolerability of bepirovirsen when dosed for 12 weeks, 12 weeks +12 weeks step-down, and 24 weeks duration in participants with CHB.

The exploratory objectives of this study are:
1. PK-PD relationships: To evaluate PK-efficacy relationship and PK-safety relationship.
2. Efficacy: To compare the efficacy between 12 weeks of bepirovirsen treatment with a loading dose or without a loading dose.
3. Efficacy: To assess the pharmacodynamic effect of bepirovirsen on exploratory viral biomarkers.
4. Virology: To assess the effect of genotype/phenotype and presence of baseline polymorphisms within the bepirovirsen binding site to assess the effect on treatment response; To assess the emergence of mutations within the GSK binding site, and elsewhere in the hepatitis B genome, during and after treatment.
5. Immunology: To assess the effect of 12 weeks, 12 weeks+12 weeks step-down or 24 weeks treatment with bepirovirsen on immunological biomarkers; To describe the relationship(s) between virology biomarkers, including but not limited to HBsAg, and immunological biomarkers.
6. Patient Reported Outcomes: To assess changes from baseline in patient reported outcomes following 12 weeks, 12 weeks+12 weeks step-down, and 24 weeks of treatment with bepirovirsen.

### C. Study Population: Inclusion and Exclusion Criteria

The inclusion criteria of this study include:
1. At least 18 years of age at the time of signing the informed consent.
2. Participants who have documented chronic HBV infection ≥6 months prior to screening AND
   a. Not currently on nucleos(t)ide analogue therapy population defined as participants who never received HBV treatment (treatment naïve) OR must have ended nucleos(t)ide therapy at least 6 months prior to the screening visit;
   b. OR Currently receiving stable nucleos(t)ide analogue therapy population defined as no changes to their nucleos(t)ide regimen from at least 6 months prior to screening and with no planned changes to the stable regimen over the duration of the study.
3. Plasma or serum HBsAg concentration>100 IU/mL.
4. Plasma or serum HBV DNA concentration.
   a. Participants not currently on nucleos(t)ide analogue therapy, plasma or serum HBV DNA>2000 IU/mL;
   b. Participants who are receiving stable nucleos(t)ide analogue therapy must be adequately suppressed, defined as plasma or serum HBV DNA<90 IU/mL.
5. Alanine Transaminase (ALT)
   a. ALT for treatment naïve participants and for participants who are not currently receiving treatment;
      i. ALT<3×Upper Limit of Normal (ULN) will be included initially
         1. If agreed by the IDMC after review of safety data, the ALT inclusion criteria may be expanded to include participants with ALT<5×ULN.
   b. ALT≤2×ULN for participants who are receiving stable nucleos(t)ide analogue therapy

The exclusion criteria of this study include:
1. Clinically significant abnormalities, aside from chronic HBV infection in medical history (e.g., moderate-severe liver disease other than chronic HBV, acute coronary syndrome within 6 months of screening, major surgery within 3 months of screening, significant/unstable cardiac disease, uncontrolled diabetes, bleeding diathesis or coagulopathy) or physical examination.
2. Co-infection with:
   a. Current or past history of Hepatitis C virus (HCV);
   b. Human immunodeficiency virus (HIV);
   c. Hepatitis D virus (HDV).
3. History of or suspected liver cirrhosis and/or evidence of cirrhosis as determined by
   a. both Aspartate aminotransferase (AST)-Platelet Index (APRI) >2 and FibroSure/FibroTest result >0.7.
      i. If only one parameter (APRI or FibroSure/FibroTest) result is positive, a discussion with the Medical Monitor is required before inclusion in study is permitted.
   b. Regardless of APRI of Fibrosure/FibroTest score, if the participant meets one of the following historical criteria, they will be excluded from the study.
   i. Liver biopsy (i.e., Metavir Score F4).
      ii. Liver stiffness >12 kPa.
4. Diagnosed or suspected hepatocellular carcinoma as evidenced by the following
   a. Alpha-fetoprotein concentration≥200 ng/mL;
   b. If the screening alpha fetoprotein concentration is ≥50 ng/mL and <200 ng/mL, the absence of liver mass must be documented by imaging within 6 months before randomization.
5. History of malignancy within the past 5 years with the exception of specific cancers that are cured by surgical resection (e.g., skin cancer). Participants under evaluation for possible malignancy are not eligible.
6. History of vasculitis or presence of symptoms and signs of potential vasculitis [e.g., vasculitic rash, skin ulceration, repeated blood detected in urine without identified cause] or history/presence of other diseases that may be associated with vasculitis condition (e.g., systemic lupus erythematosus, rheumatoid arthritis, relapsing polychondritis, mononeuritis multiplex).
7. History of extrahepatic disorders possibly related to HBV immune conditions (e.g., nephrotic syndrome, any type of glomerulonephritis, polyarteritis nodosa, cryoglobulinaemia, uncontrolled hypertension).
8. Positive (or borderline positive) ANCA at screening:
   a. Participants that meet this criteria may be considered for inclusion in the study following:
      i. Analysis of MPO-ANCA [pANCA] and PR3-ANCA [cANCA] AND
      ii. A discussion with the Medical Monitor to review participant's complete medical history to ensure no past history or current manifestations of a vasculitic/inflammatory/auto-immune condition.
9. Low C3 at screening AND evidence of past history or current manifestations of vasculitic/inflammatory/auto-immune conditions:
   a. All participants with low C3 at screening should have their medical history discussed with the Medical Monitor prior to enrolment.
10. History of alcohol or drug abuse/dependence:
   a. Current alcohol use as judged by investigator to potentially interfere with participant compliance;
   b. History of or current drug abuse/dependence as judged by the investigator to potentially interfere with participant compliance
      i. Refers to illicit drugs and substances with abuse potential. Medications that are used by the participant as directed, whether over-the-counter or through prescription, are acceptable and would not meet the exclusion criteria.
11. Currently taking, or took within 3 months of screening, any immunosuppressing drugs (e.g., prednisone), other than a short course of therapy (≤2 weeks) or topical/inhaled steroid use.
12. Participants for whom immunosuppressive treatment is not advised, including therapeutic doses of steroids, will be excluded.
13. Currently taking, or took within 12 months of screening, any interferon-containing therapy.
14. Participants requiring anti-coagulation therapies (for example warfarin, Factor Xa inhibitors or anti-platelet agents like clopidogrel).
15. The participant has participated in a clinical trial and has received an investigational product within the following time period prior to the first dosing day in the current study: 5 half-lives (if known) or twice the duration (if known) of the biological effect of the study treatment (whichever is longer) or 90 days (if half-life or duration is unknown).
16. Prior treatment with any oligonucleotide or small interfering RNA (siRNA) within 12 months prior to the first dosing day
17. Fridericia's QT correction formula (QTcF) ≥450 msec (if single electrocardiogram [ECG] at screening shows QTcF ≥450 msec, a mean of triplicate measurements should be used to confirm that participant meets exclusion criterion).
18. Laboratory results as follows
   a. Serum albumin <3.5 g/dL
   b. Glomerular filtration rate (GFR)<60 mL/min/1.73 m² as calculated by the CKD-EPI formula (for Japan, JSN-CKDI equation).
   c. INR>1.25
   d. Platelet count<140×10⁹/L
   e. Total bilirubin>1.25×ULN
      i. For participants with benign unconjugated hyperbilirubinemia with total bilirubin>1.25×ULN, discussion for inclusion to the study is required with the Medical Monitor
   f. Urine albumin to creatinine ratio (ACR) ≥0.03 mg/mg (or ≥30 mg/g). In the event of an ACR above this threshold, eligibility may be confirmed by a second measurement
      i. In cases where participants have low urine albumin and low urine creatinine levels resulting in a urine ACR calculation ≥0.03 mg/mg (or ≥30 mg/g), the investigator should confirm that the participant does not have a history of diabetes, hypertension or other risk factors that may affect renal function and discuss with the Medical Monitor, or designee.
19. History of/sensitivity to bepirovirsen or components thereof or a history of drug or other allergy that, in the opinion of the investigator or Medical Monitor, contraindicates their participation.

### D. Study Assessments and Procedures

The primary objective measurements for efficacy include HBsAg and HBV DNA. The primary efficacy endpoint is sustained virologic response (SVR), which is a composite endpoint defined as HBsAg<LLOQ and HBV DNA<LLOQ at the end of bepirovirsen treatment which is sustained for 24 weeks post-bepirovirsen treatment. Seroclearance refers to participants with HBsAg and HBV DNA<LLOQ (with or without the formation of HBs-antibody). Seroconversion refers to participants with HBsAg and HBV DNA<LLOQ plus formation of HBs-antibody. Both terms are used to evaluate efficacy. For the purposes of this study, sustained response is defined as a continuous 24 weeks from end of bepirovirsen treatment during which levels of HBsAg in serum remain less than LLOQ and HBV DNA less than LLOQ.

Any HBsAg greater than LLOQ or HBV DNA greater than LLOQ after achieving HBsAg seroclearance and HBV DNA suppression needs to be confirmed by re-test within 1 week of receiving the test result. The re-test result will be used if the first test is not confirmed.

Safety assessments are conducted at planned time points during the course of the study, and additional time points for safety tests may be added based on newly available data to ensure appropriate safety monitoring. Safety assessments include physical examinations, injection site reactions, vital signs, electrocardiograms, and clinical safety laboratory assessment.

Adverse events (AEs) and serious adverse events (SAEs) are detected, documents, and reported. An adverse event is any untoward medical occurrence in a clinical study participant, temporally associated with the use of a study intervention, whether or not considered related to the study intervention. An AE can therefore be any unfavorable and unintended sign (including an abnormal laboratory finding), symptom, or disease (new or exacerbated) temporally associated with the use of a study intervention. A serious adverse event is defined as any untoward medical occurrence that, at any dose: results in death or is life-threatening.

Blood samples are collected for measurement of plasma concentrations of bepirovirsen and/or nucleos(t)ide. Pharmacodynamic parameters explored in this study include but are not limited to:
Change from baseline: HBsAg, HBV DNA, HBeAg, and HBsAb levels.
Time to event: virologic response, nadir of HBsAg and HBV DNA, seroclearance (HBsAg), HBV DNA level below LLOQ, seroconversion (HBsAb and HBeAb); peak of ALT flares.
Safety assessments including but not limited to vital signs, electrocardiograms, laboratory measurements and adverse events.

Collection of samples for other biomarker research is also part of this study. These exploratory biomarker samples were collected to evaluate the pathogenesis of CHB; the absorption, distribution, metabolism, or excretion of bepirovirsen; or the participant's response to bepirovirsen. In addition, continuing research may identify other proteins, transcripts or biomarkers related to bepirovirsen treatment, the response to bepirovirsen or the pathogenesis of CHB, which are evaluated in these samples.

### Example 2

### Interim Analysis Results of the Phase 2b Clinical Study

At the time of filing the first priority patent application (U.S. 63/324,662), the Phase 2b clinical trial was ongoing, and interim analysis (IA) results are discussed below. The demographic characteristics of participants in four arms for both ON-NUC and NOT ON-NUC cohorts are shown in Tables 1 and 2, respectively.

TABLE 1
Demographic characteristics of participants in four arms for ON-NUC cohorts.

| | Arm 1 | | Arm 2 | | Arm 3 | | Arm 4 | |
|---|---|---|---|---|---|---|---|---|
| | (N = 68) | | (N = 68) | | (N = 68) | | (N = 23) | |
| Sex Male, n (%) | 48 | (71%) | 49 | (72%) | 51 | (75%) | 17 | (74%) |
| Age (Years), Mean | 49.0 | (11.54) | 46.1 | (12.60) | 47.4 | (11.18) | 49.8 | (11.21) |

(SD)

Race, n (%)

| American Indian or | 0 | | 0 | | 1 | (1%) | 0 | |
|---|---|---|---|---|---|---|---|---|
| Alaska native | | | | | | | | |
| Asian | 36 | (53%) | 35 | (51%) | 36 | (53%) | 12 | (52%) |
| Black or African | 2 | (3%) | 1 | (1%) | 4 | (6%) | 0 | |
| American | | | | | | | | |
| White | 30 | (44%) | 32 | (47%) | 26 | (38%) | 11 | (48%) |
| Mixed race | 0 | | 0 | | 1 | (1%) | 0 | |
| BMI (kg/m\hat{ }2) <30, | 63 | (93%) | 61 | (90%) | 67 | (99%) | 23 | (100%) |

n (%)

TABLE 2
Demographic characteristics of participants in four arms for NOT ON-NUC cohorts.

| | Arm 1 | | Arm 2 | | Arm 3 | | Arm 4 | |
|---|---|---|---|---|---|---|---|---|
| | (N = 70) | | (N = 68) | | (N = 68) | | (N = 24) | |
| Sex Male (%) | 33 | (47%) | 41 | (60%) | 39 | (57%) | 11 | (46%) |
| Age (Years) Mean (SD) | 44.5 | (11.10) | 43.8 | (9.92) | 40.7 | (11.10) | 42.4 | (12.02) |

Race N (%)

| Asian | 37 | (53%) | 44 | (65%) | 38 | (56%) | 12 | (50%) |
|---|---|---|---|---|---|---|---|---|
| Black or African | 9 | (13%) | 4 | (6%) | 6 | (9%) | 1 | (4%) |
| American | | | | | | | | |
| White | 24 | (34%) | 20 | (29%) | 24 | (35%) | 11 | (46%) |
| Mixed race | 0 | | 0 | | 0 | | 0 | |
| BMI (kg/m\hat{ }2) <30, n | 61 | (87%) | 61 | (90%) | 63 | (93%) | 21 | (88%) |

(%)

The disease characteristics of participants in four arms for both ON-NUC and NOT ON-NUC cohorts are shown in Tables 3 and 4, respectively.

TABLE 3
Disease characteristics for the participants in four arms for ON-NUC cohorts.

| | Arm 1 | | Arm 2 | | Arm 3 | | Arm 4 | |
|---|---|---|---|---|---|---|---|---|
| | (N = 68) | | (N = 68) | | (N = 68) | | (N = 23) | |
| HBsAg (log10 IU/mL), | 3.322 | | 3.289 | | 3.315 | | 3.348 | |
| Median | | | | | | | | |
| low (<=3 log10 | 19 | (28%) | 23 | (34%) | 19 | (28%) | 3 | (13%) |
| IU/mL), n (%) | | | | | | | | |
| HBeAg negative, n (%) | 50 | (74%) | 47 | (70%) | 44 | (65%) | 16 | (70%) |
| ALT <= ULN, n (%) | 62 | (91%) | 60 | (90%) | 62 | (91%) | 21 | (91%) |

TABLE 4
Disease characteristics for the participants
in four arms for NOT ON-NUC cohorts.

| | Arm 1 | | Arm 2 | | Arm 3 | | Arm 4 | |
|---|---|---|---|---|---|---|---|---|
| | (N = 70) | | (N = 68) | | (N = 68) | | (N = 24) | |
| HBsAg (log10 IU/mL), | 3.683 | | 3.602 | | 3.742 | | 3.879 | |
| Median | | | | | | | | |
| low (<=3 log10 | 12 | (17%) | 15 | (22%) | 11 | (16%) | 5 | (21%) |
| IU/mL), n (%) | | | | | | | | |
| HBeAg negative, n (%) | 49 | (70%) | 52 | (76%) | 52 | (76%) | 17 | (71%) |
| ALT <= ULN, n (%) | 50 | (71%) | 48 | (71%) | 47 | (69%) | 15 | (63%) |

The disposition of participants (Intent-to-Treat population) in four arms for both ON-NUC and NOT ON-NUC cohorts are shown in Tables 5 and 6, respectively.

TABLE 5
Disposition of participants in four arms for ON-NUC cohorts.

| | | | |
|---|---|---|---|
| Arm 1 | Arm 2 | Arm 3 | Arm 4 |
| N = 68 | N = 68^{#} | N = 68 | N = 23 |

Treatment Status

| Ongoing | 0 | | 0 | | 1* | (1%) | 0 | |
|---|---|---|---|---|---|---|---|---|
| Completed | 64 | (94%) | 63 | (93%) | 64 | (94%) | 22 | (96%) |
| Prematurely discontinued | 4 | (6%) | 4 | (6%) | 3 | (4%) | 1 | (4%) |

Primary reason for treatment discontinuation

| | | | | | | | |
|---|---|---|---|---|---|---|---|
| Adverse event | 1 | (1%) | 2 | (3%) | 2 | (3%) | 0 |

| | | | | | |
|---|---|---|---|---|---|
| Physician decision | 0 | 1 | (1%) | 0 | 0 |
| Subject reached protocol- | 0 | 1 | (1%) | 0 | 0 |

| defined stopping criteria | | | | | | | |
|---|---|---|---|---|---|---|---|
| Withdrawal by subject | 3 | (4%) | 0 | 1 | (1%) | 1 | (4%) |

^{#}Subject was randomised but never received a dose so treatment status numbers do not sum to N
*Subject has treatment status ongoing due to missing end of treatment assessment at the time of the IA data cut.

TABLE 6
Disposition of participants in four arms for NOT ON-NUC cohorts.

| | | | |
|---|---|---|---|
| Arm 1 | Arm 2 | Arm 3 | Arm 4 |
| N = 70 | N = 68 | N = 68 | N = 24 |

Treatment Status

| Ongoing | 0 | | 1 | (1%) | 0 | | 0 | |
|---|---|---|---|---|---|---|---|---|
| Completed | 62 | (89%) | 64 | (94%) | 60 | (88%) | 21 | (88%) |
| Prematurely discontinued | 8 | (11%) | 2 | (3%) | 8 | (12%) | 3 | (13%) |

Primary reason for treatment discontinuation

| Adverse event | 3 | (4%) | 0 | | 5 | (7%) | 0 | |
|---|---|---|---|---|---|---|---|---|
| Physician decision | 0 | | 1 | (1%) | 0 | | 1 | (4%) |
| Subject reached protocol- | 1 | (1%) | 0 | | 1 | (1%) | 1 | (4%) |
| defined stopping criteria | | | | | | | | |
| Withdrawal by subject | 4 | (6%) | 1 | (1%) | 2 | (3%) | 1 | (4%) |

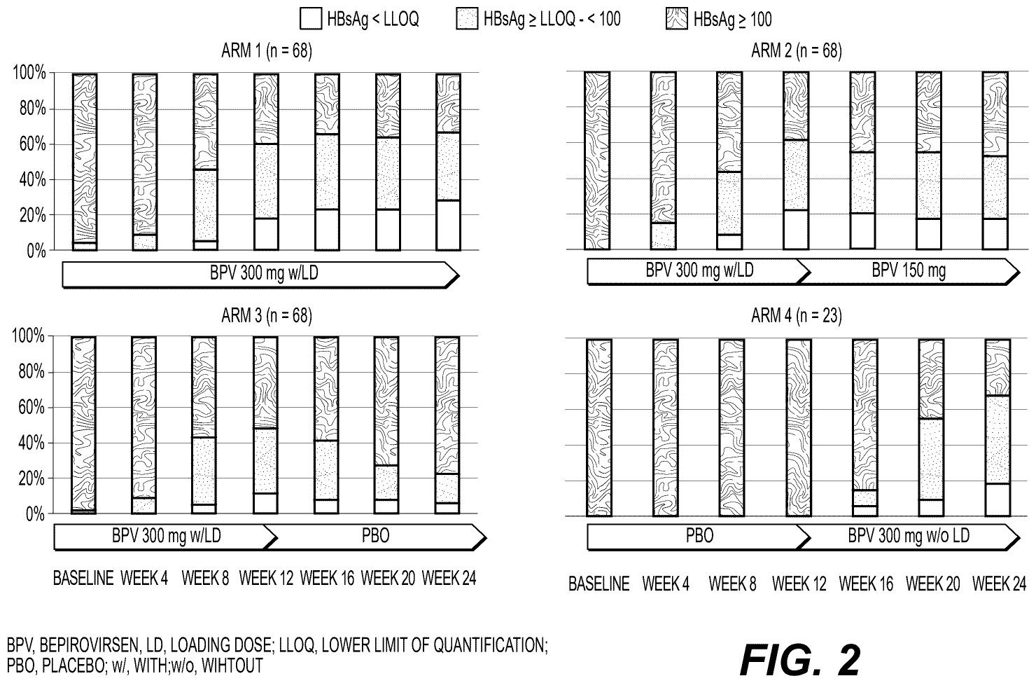

For ON-NUC cohorts, 227 patients (73% male, 52% Asian, 69% HBeAg negative, 28% HBsAg≤3 log 10 IU/mL) were included in the intent-to-treat population (68, 68, 68 and 23 patients, respectively, in arms 1-4); 12 patients (5%) discontinued treatment. At the end of treatment, 28%, 17%, 90% and 14% patients had HBsAg<LLOQ and HBV DNA<LLOQ in arms 1-4, respectively. HBsAg response data are shown in FIG. 2.

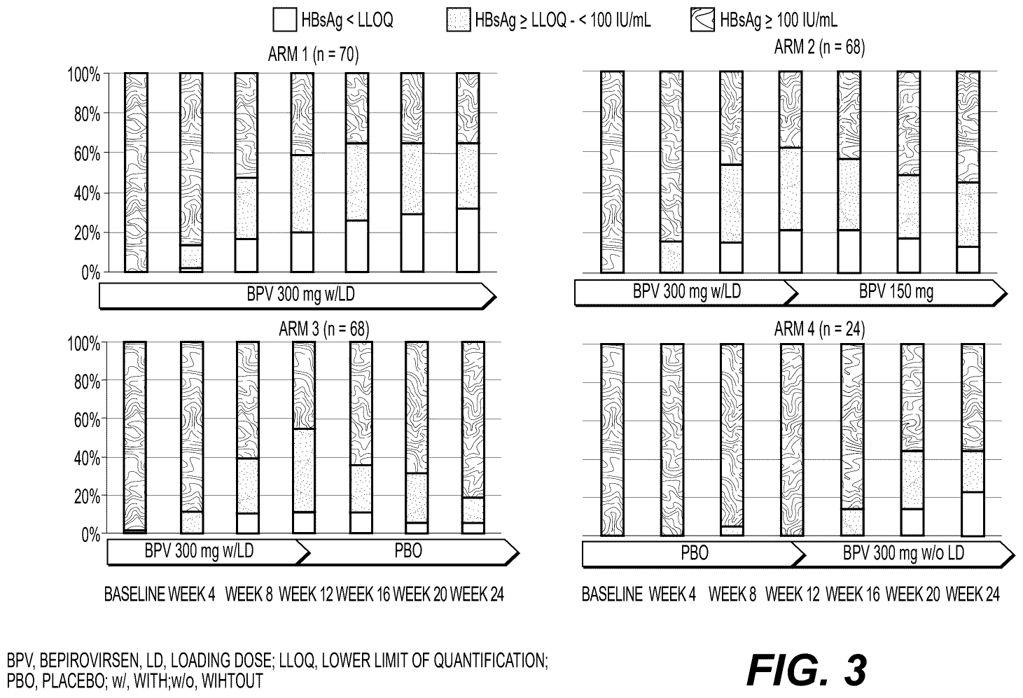

For NOT ON-NUC cohorts, 230 patients (54% male, 57% Asian, 74% HBeAg negative, 19% HBsAg<3 log 10 IU/mL) were included in the intent-to-treat population (70, 68, 68 and 24 patients, respectively, in arms 1-4); 21 patients (9%) discontinued treatment. At the end of treatment, 29%, 13%, 7% and 0% patients had HBsAg<LLOQ and HBV DNA<LLOQ in arms 1-4, respectively. HBsAg response data are shown in FIG. 3.

Efficacy of the bepirovirsen treatment was evaluated based on the interim data. Virologic response at the end of treatment is defined as achieving HBsAg<LLOQ and HBV DNA<LLOQ in the absence of rescue medication in the end of treatment visit window. SVR success is defined as HBsAg<LLOQ and HBV DNA<LLOQ for 24 weeks after the planned end of bepirovirsen treatment in the absence of rescue medication.

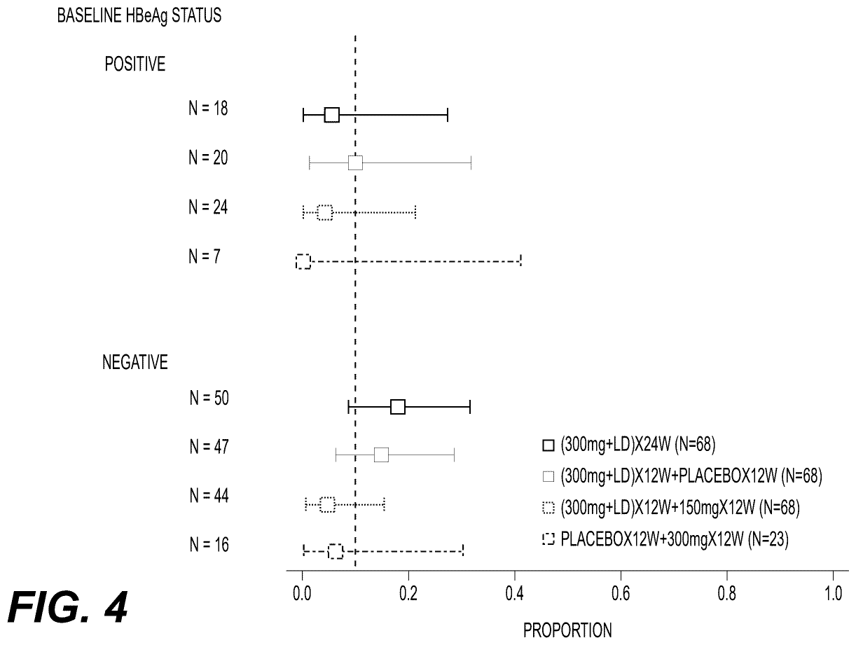

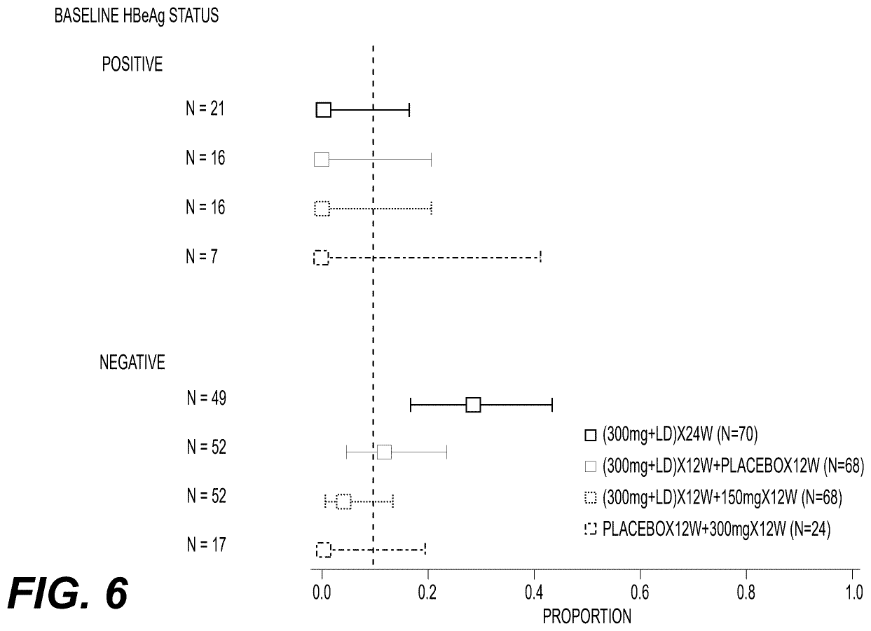

Virologic response at the end of treatment by subgroup—baseline HBeAg status was analyzed as a secondary endpoint. For ON-NUC cohorts, no apparent difference in virologic response in the end of treatment analysis window in patients with HBeAg status positive and negative. See FIG. 4. The dashed line represents the overall virologic response rate at the end of treatment across all treatment arms. For NOT ON-NUC cohorts, patients with negative HBeAg status appear to have a slightly higher chance of achieving virologic response at the end of the treatment. See FIG. 6. Two SVR responders were both HBeAg negative at baseline.

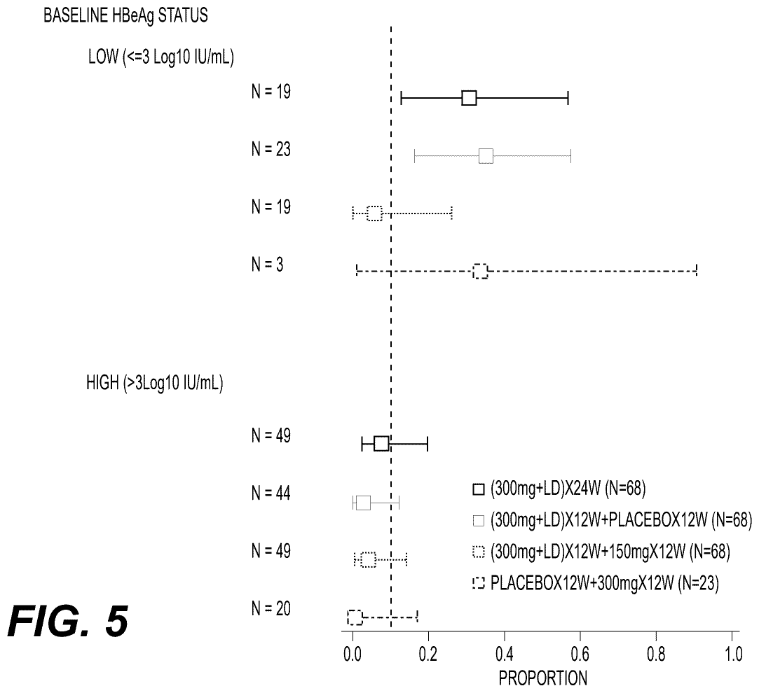

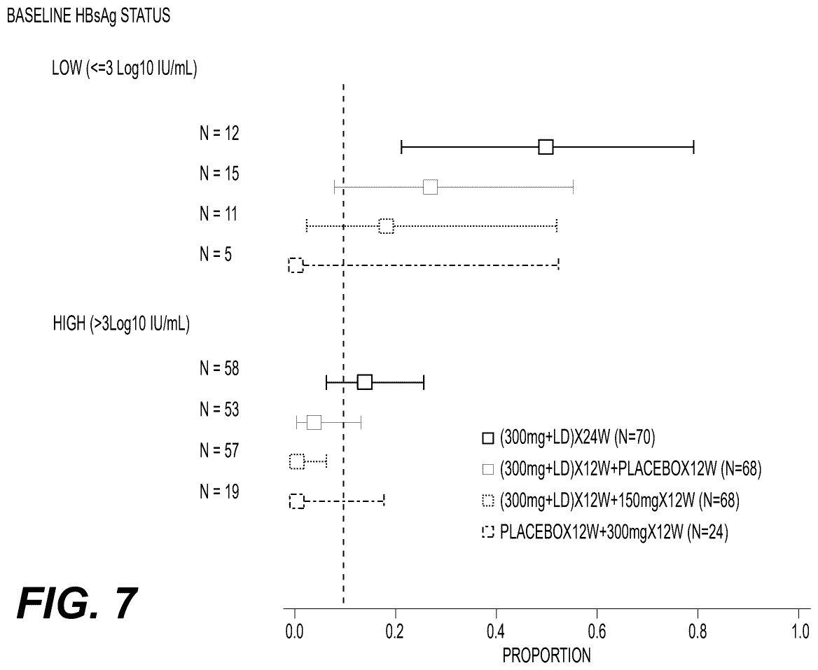

Virologic response at the end of treatment by subgroup—baseline HBsAg status was also analyzed as a secondary endpoint. For ON-NUC cohorts, proportion of subjects with virologic response at the end of treatment is higher for patients with a low HBsAg baseline level (≤3 Log 10 IU/mL) as compared to those with a high HBsAg baseline level (>3 Log 10 IU/mL). See FIG. 5. Six out of eight SVR responders had a low HBsAg baseline level (≤3 Log 10 IU/mL). For NOT ON-NUC cohorts, proportion of subjects with virologic response at the end of treatment is also higher for patients with a low HBsAg baseline level (≤3 Log 10 IU/mL). See FIG. 7. Two SVR responders both had a low HBsAg baseline level (≤3 Log 10 IU/mL).

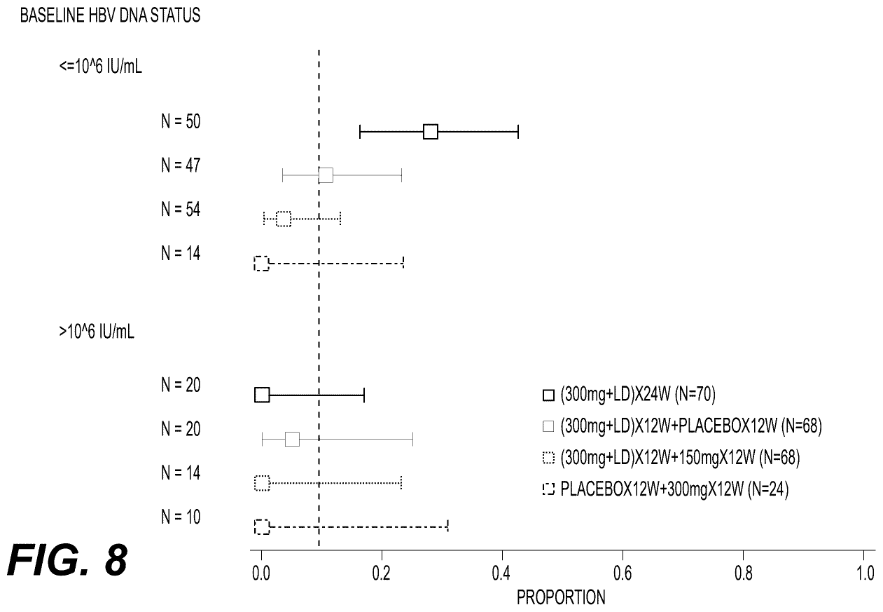

In addition, for NOT ON-NUC cohorts, virologic response at the end of treatment by subgroup—baseline HBV DNA status was analyzed as a secondary endpoint. See FIG. 8.

Receiver Operating Charateristic (ROC) tables were used to compare different cuts of the data for increasing values of baseline HBsAg. The ROC tables can be used to assess each “cut” in terms of the inclusion of subjects who are responders, against the exclusion of subjects who are non-responders. See Tables 7 and 8 for example.

TABLE 7
Summary of ROC Analysis for Baseline HBsAg (log10
IU/mL) as a Predictor of Virologic Response
at the End of Treatment (ON-NUC, arm 1).

| HBsAg Cut-off | Specificity | Sensitivity | |
|---|---|---|---|
| (log10 IU/mL) | (1-False positive rate) | (True positive rate) | Accuracy |
| 1.6 | 0.98 | 0.00 | 0.838 |
| 2.0 | 0.98 | 0.30 | 0.882 |
| 2.1 | 0.98 | 0.30 | 0.882 |
| 2.2 | 0.95 | 0.30 | 0.853 |
| 2.3 | 0.95 | 0.30 | 0.853 |
| 2.4 | 0.95 | 0.30 | 0.853 |
| 2.5 | 0.93 | 0.30 | 0.838 |
| 2.6 | 0.91 | 0.40 | 0.838 |
| 2.7 | 0.91 | 0.50 | 0.853 |
| 2.8 | 0.84 | 0.50 | 0.794 |
| 2.9 | 0.78 | 0.60 | 0.750 |
| 3.0 | 0.76 | 0.70 | 0.750 |
| 3.1 | 0.69 | 0.70 | 0.691 |
| 3.2 | 0.60 | 0.70 | 0.618 |
| 3.3 | 0.52 | 0.80 | 0.559 |
| 3.4 | 0.45 | 0.90 | 0.515 |
| 3.5 | 0.38 | 0.90 | 0.456 |
| 3.6 | 0.31 | 0.90 | 0.397 |
| 3.7 | 0.21 | 0.90 | 0.309 |
| 3.8 | 0.16 | 1.00 | 0.279 |
| 3.9 | 0.14 | 1.00 | 0.265 |
| 4.0 | 0.14 | 1.00 | 0.265 |
| 4.1 | 0.10 | 1.00 | 0.235 |
| 4.2 | 0.09 | 1.00 | 0.221 |
| 4.3 | 0.07 | 1.00 | 0.206 |
| 4.4 | 0.03 | 1.00 | 0.176 |
| 4.5 | 0.02 | 1.00 | 0.162 |
| 4.6 | 0.00 | 1.00 | 0.147 |

TABLE 8
Summary of ROC Analysis for Baseline HBsAg (log10
IU/mL) as a Predictor of Virologic Response at
the End of Treatment (NOT ON-NUC, arm 1).

| HBsAg Cut-off | Specificity | Sensitivity | |
|---|---|---|---|
| (log10 IU/mL) | (1-False positive rate) | (True positive rate) | Accuracy |
| 2.0 | 1.00 | 0.07 | 0.814 |
| 2.3 | 0.96 | 0.14 | 0.800 |
| 2.4 | 0.96 | 0.29 | 0.829 |
| 2.5 | 0.95 | 0.29 | 0.814 |
| 2.6 | 0.95 | 0.29 | 0.814 |
| 2.7 | 0.93 | 0.29 | 0.800 |
| 2.8 | 0.91 | 0.36 | 0.800 |
| 2.9 | 0.91 | 0.36 | 0.800 |
| 3.0 | 0.89 | 0.57 | 0.829 |
| 3.1 | 0.89 | 0.57 | 0.829 |
| 3.2 | 0.88 | 0.64 | 0.829 |
| 3.3 | 0.82 | 0.64 | 0.786 |
| 3.4 | 0.80 | 0.79 | 0.800 |
| 3.5 | 0.70 | 0.79 | 0.714 |
| 3.6 | 0.64 | 0.93 | 0.700 |
| 3.7 | 0.57 | 1.00 | 0.657 |
| 3.8 | 0.52 | 1.00 | 0.614 |
| 3.9 | 0.48 | 1.00 | 0.586 |
| 4.0 | 0.39 | 1.00 | 0.514 |
| 4.1 | 0.36 | 1.00 | 0.486 |
| 4.2 | 0.30 | 1.00 | 0.443 |
| 4.3 | 0.23 | 1.00 | 0.386 |
| 4.4 | 0.20 | 1.00 | 0.357 |
| 4.5 | 0.20 | 1.00 | 0.357 |
| 4.6 | 0.20 | 1.00 | 0.357 |
| 4.7 | 0.16 | 1.00 | 0.329 |
| 4.8 | 0.07 | 1.00 | 0.257 |
| 4.9 | 0.05 | 1.00 | 0.243 |
| 5.0 | 0.04 | 1.00 | 0.229 |

### Example 3

### Mechanistic Pharmacokinetics/Pharmacodynamics Modelling of the Simultaneous Effects of Bepirovirsen on Hepatitis B Surface Antigen (HBsAg) and Alanine Transaminase (ALT) Changes in Chronic Hepatitis B (CHB) Patients

Following treatment with bepirovirsen, decreases in HBsAg were often observed prior to or in parallel with transient increases in alanine transaminase (ALT), suggesting an increase in immune-mediated clearance of infected hepatocytes and subsequent release of ALT. A mechanistic Pharmacokinetics (PK)/Pharmacodynamics (PD) model was developed to describe the time course of bepirovirsen systemic exposures and its effect on changes in HBsAg and ALT and to identify patient characteristics that may be predictors of exposure and response to treatment.

PK, HBsAg and ALT data were pooled from a Phase 1 study (GSK Study 213725) (n=28, dose range 75-450 mg) in healthy volunteers, Phase 2a (GSK Study 205695) and Phase 2b (GSK Study 209668) studies (n=31 and 455, respectively, doses of 150 or 300 mg) in participants with CHB. Bepirovirsen was administrated subcutaneously for up to 4 weeks in the Phase 1 and 2a studies and 12 or 24 weeks in the Phase 2b study. In the developed PK/PD model, HBsAg levels below the lower limit of quantification (LLOQ, <0.05 IU/mL) were estimated using a likelihood-based approach in order to predict a complete HBsAg profile during on- and off-treatment periods. A threshold for continued viral suppression was implemented to reflect an increased probability of participants achieving and maintaining HBsAg levels below LLOQ (BLQ) at end of study if their predicted HBsAg levels fall below the threshold. Direct and indirect drug effects on ALT were explored to describe ALT increases. The indirect effect was driven through reduction of HBsAg, resulting in immune-mediate hepatocyte senescence and subsequent ALT release. Using the developed PK/PD model, effects of bepirovirsen loading dose (LD), as well as baseline patient characteristics (e.g. demographics, HBsAg, HBeAg, genotype), on the response to bepirovirsen treatment were evaluated.

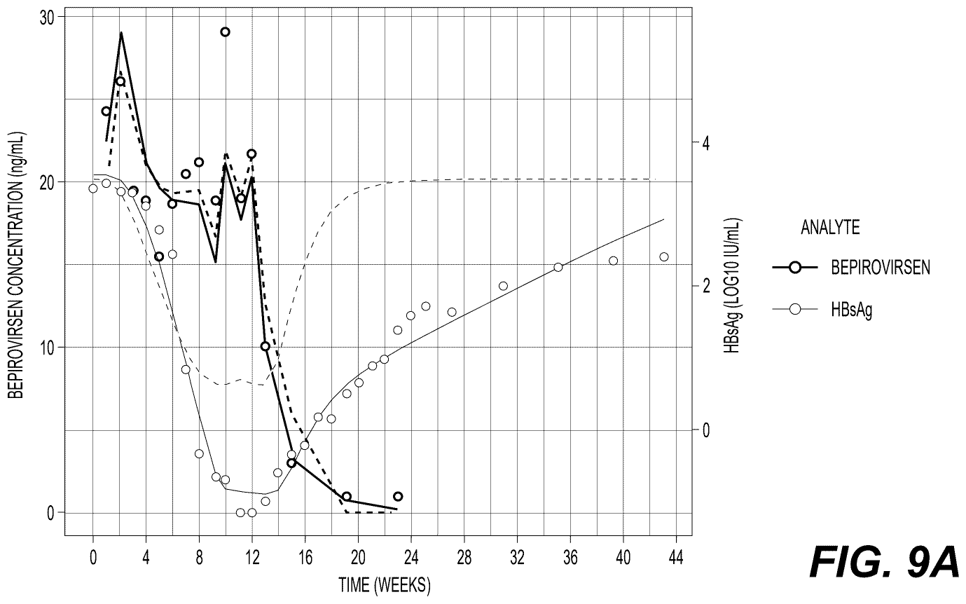

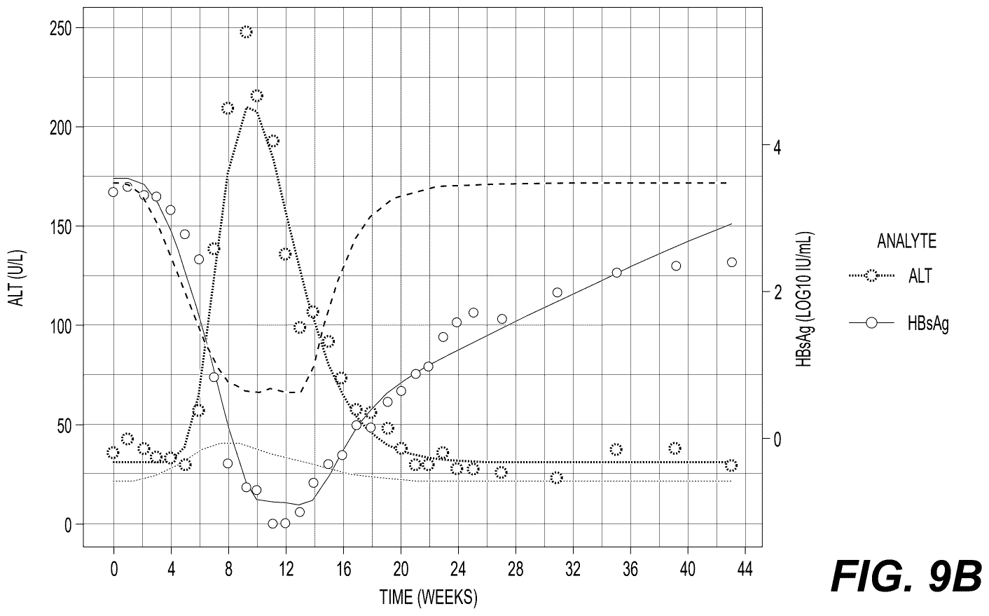

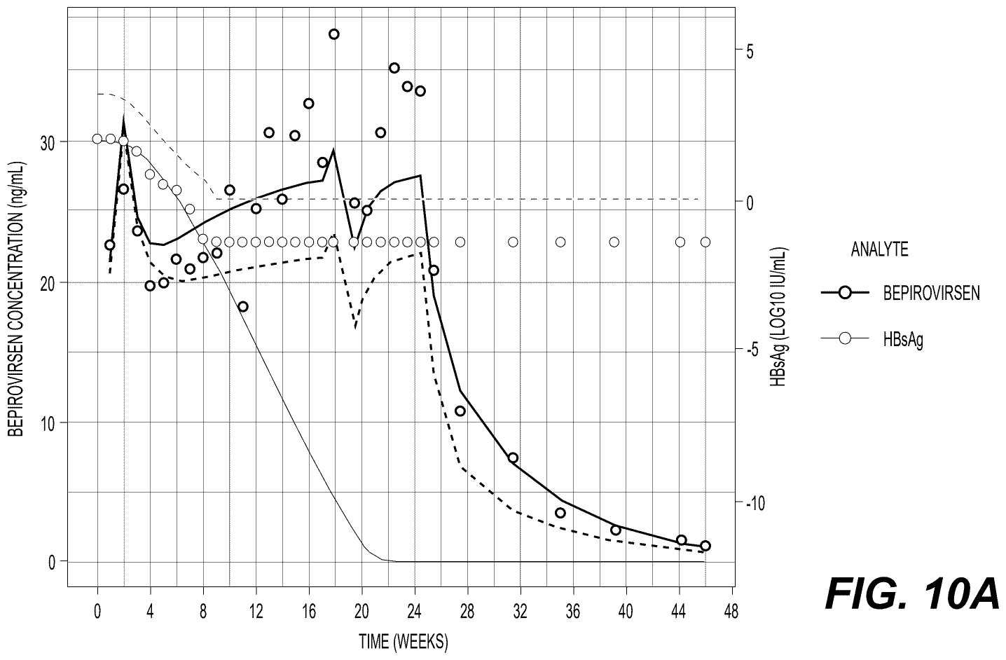

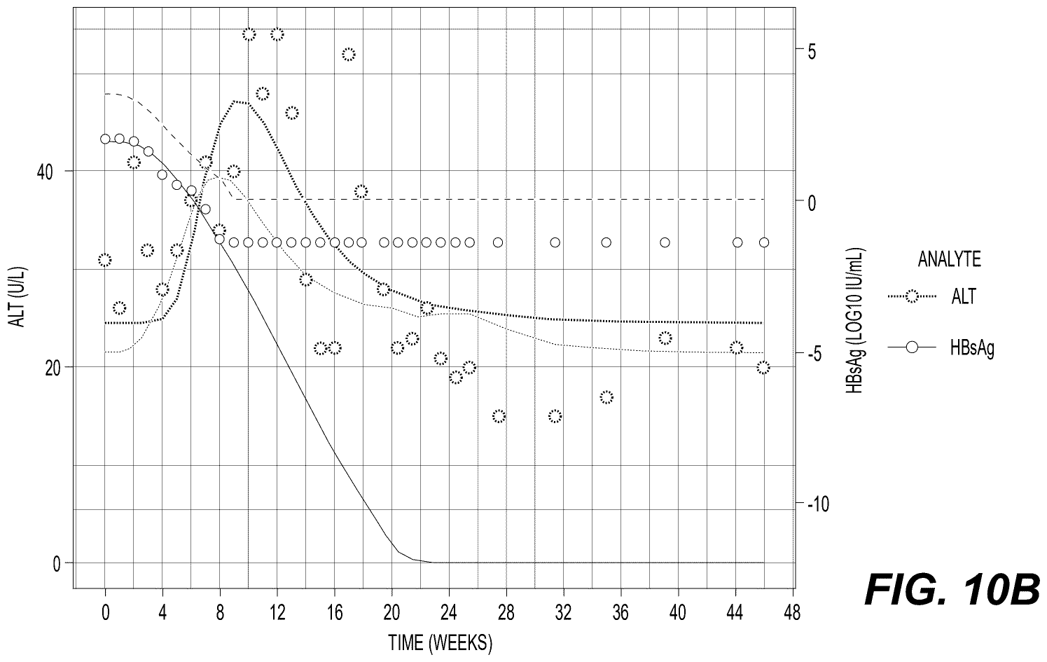

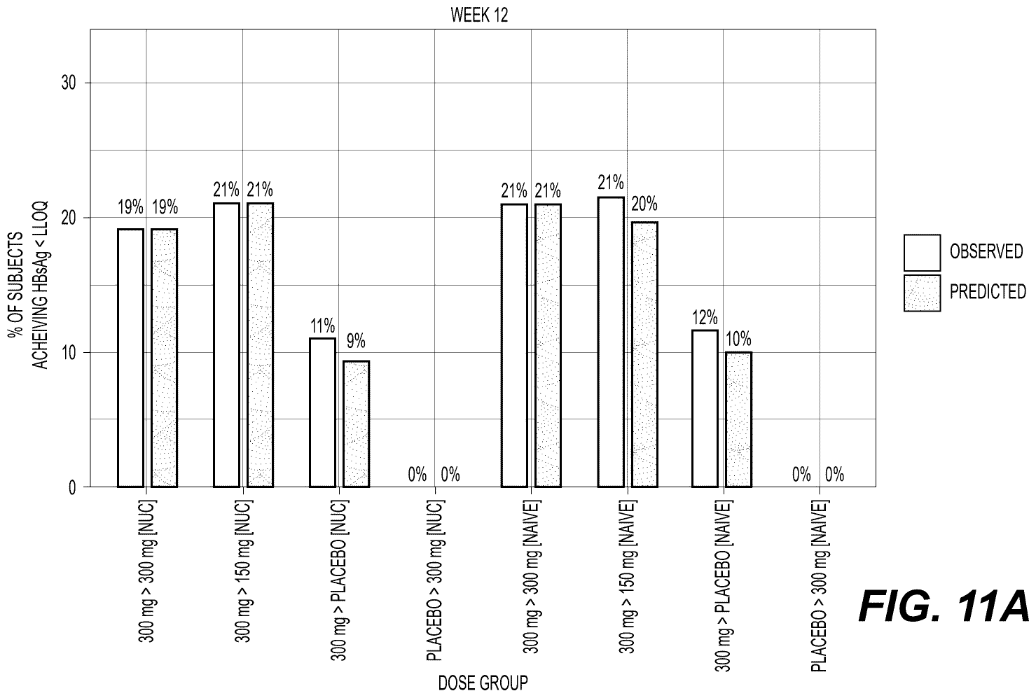

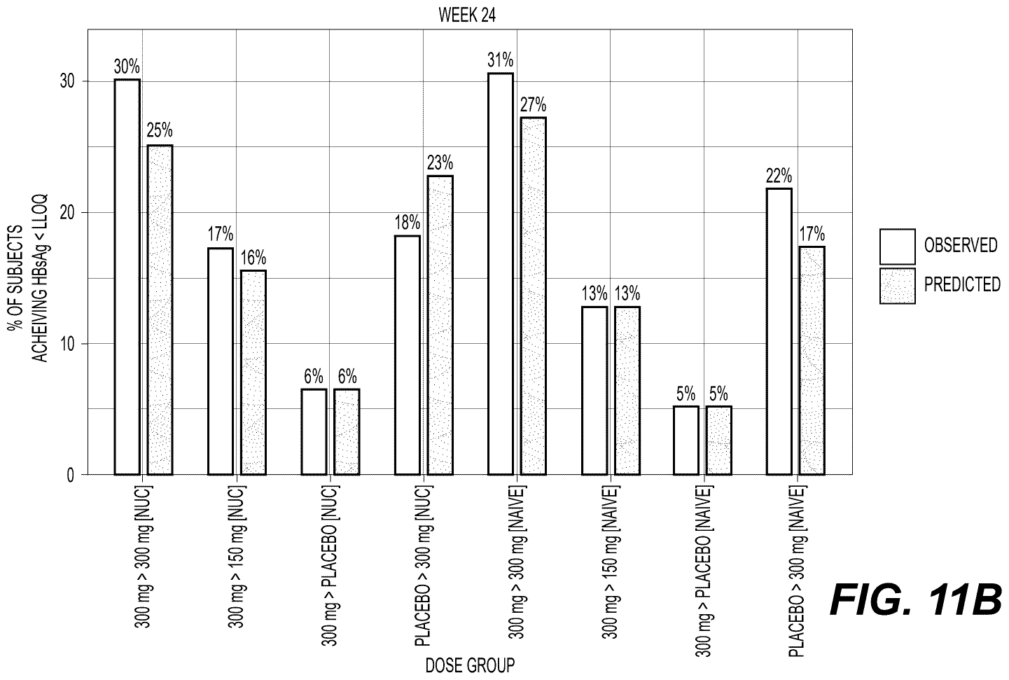

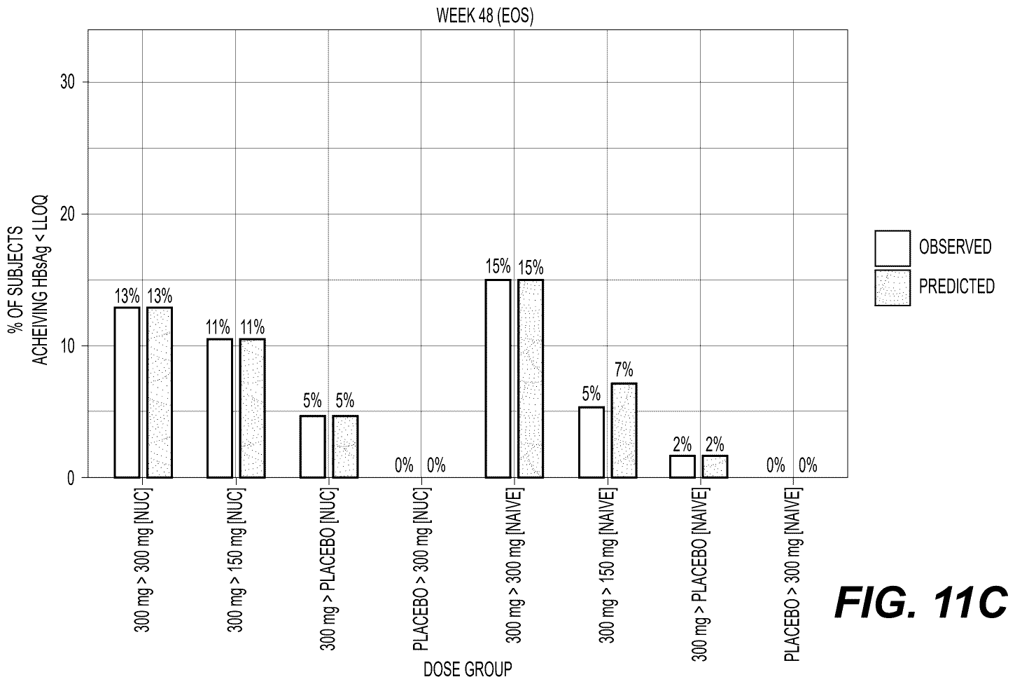

The model accurately captured the time-course of bepirovirsen exposure, HBsAg and ALT changes. The increase of ALT was primarily driven by HBsAg suppression rather than a direct drug effect and baseline HBsAg was identified as a significant predictor of response to treatment. Model results indicate no significant efficacy benefit with inclusion of LD in the first two weeks of treatment; nor a difference in exposure-response relationship based on nucleos(t)ide coadministration. Examples of observed and model-predicted individual subject PK, HBsAg, and ALT profiles are shown in FIGS. 9A, 9B, 10A, and 10B (Dots represent observed data, dashed and solid lines represent population and individual subject predictions based on the PK/PD model). The model predicted the percentage of subjects who achieved HBsAg<LLOQ at 12 weeks, at 24 weeks, and at 48 weeks (during the off-treatment period) (FIGS. 11A, 11B and 11C).

The developed PK/PD model provides a useful tool for informing key decisions regarding Phase 3 design, such as dose selection, treatment duration, and patient population. In simulations, patients with low baseline HBsAg levels were predicted to be more likely to achieve HBsAg<LLOQ following administration of bepirovirsen (BPV) compared with the overall population (HBsAg<3000 IU/mL range: 30.1%-40.0% and 12.9%-16.5% at EOT and EOS, respectively; overall population range: 20.1%-26.1% and 9.2%-10.7% at EOT and EOS, respectively) across different BPV dose regimens (Table 9). Similar response rates were predicted with and without loading doses. A higher proportion of patients were predicted to achieve a response with 24-week versus 12-week BPV treatment (39.9% vs 30.1% and 16.3% vs 12.9% at EOT and EOS, respectively) in the low baseline HBsAg population (Table 9).

TABLE 9
Summary of simulation-based HBsAg response by dose level

| | | | Participants with HBsAg | |
|---|---|---|---|---|
| Bepirovirsen dosing | | | response (%) | |
| regimen | Population | Outcome | EOT | EOS |
| 300 mg weekly × 24 | Overall | <LLOQ | 26.1 | 10.7 |
| weeks with loading dose | Baseline HBsAg | <LLOQ | 40.0 | 16.5 |
| on Day 4 and 11 | <3000 IU/mL | | | |
| 300 mg weekly × 24 | Overall | <LLOQ | 26.1 | 10.6 |
| weeks without loading | Baseline HBsAg | <LLOQ | 39.9 | 16.3 |
| dose | <3000 IU/mL | | | |
| 300 mg weekly × 12 | Overall | <LLOQ | 20.1 | 9.2 |
| weeks, without loading | Baseline HBsAg | <LLOQ | 30.1 | 12.9 |
| dose | <3000 IU/mL | | | |
| 300 mg weekly × 12 | Overall | <LLOQ | 22.3 | 10.0 |
| weeks, then 150 mg | Baseline HBsAg | <LLOQ | 35.6 | 15.1 |
| weekly × 12 weeks | <3000 IU/mL | | | |
| (without LD) | | | | |

LLOQ = −1.3 log₁₀ IU/mL (0.05 IU/mL).
Abbreviations:
EOT = end of bepirovirsen treatment (Week 12 or Week 24);
EOS = end of study (Week 60 or Week 72);
LLOQ = lower limit of quantification

### Example 4

### End of Study Results of the Phase 2b Clinical Study

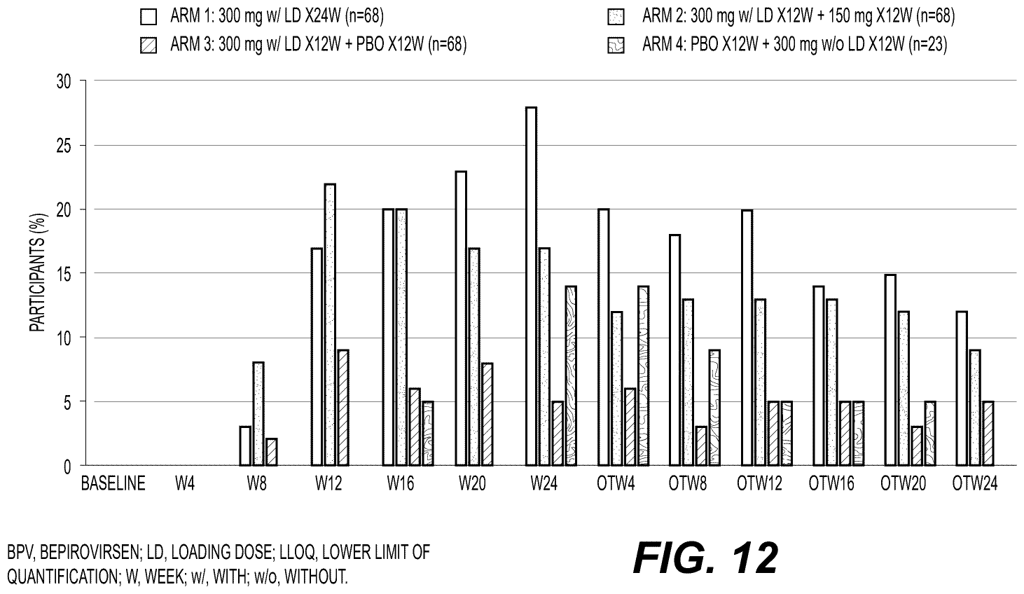

For ON-NUC cohorts, 227 patients (73% male, 52% Asian, 69% HBeAg negative, 28% HBsAg≤3 log 10 IU/mL) were included in the intent-to-treat population (68, 68, 68 and 23 patients in arms 1-4, respectively). Virologic response (VR) up to 24 weeks post-BPV treatment was achieved in 6 (9%), 6 (9%), 2 (3%) and 0 patients in arms 1-4, respectively. VR data over time are shown in FIG. 12. The patients with low baseline HBsAg levels (≤3 log 10 IU/mL) were more likely to have SVR (14%) as compared to the patients with high baseline HBsAg levels (>3 log 10 IU/mL; 3%). 10 HBeAg negative patients in arms 1-3 had sustained VR while 4 of HBeAg positive patients did in arms 1-3.300 mg BPV ×24 weeks (arm 1) and 300 mg BPV ×12 weeks+150 mg BPV ×12 weeks (arm 2) resulted in the highest proportion of patients with SVR 24 weeks off BPV treatment. See Table 10.

TABLE 10
Proportion of Participants Achieving End of Treatment Response
and Primary Endpoint at End of Study (On-NUC Population)

| | | | |
|---|---|---|---|
| Arm 1 | Arm 2 | Arm 3 | Arm 4 |

End of bepirovirsen treatment

| | | | | | | | | |
|---|---|---|---|---|---|---|---|---|
| Overall | 16/68 | (24%) | 9/67 | (13%) | 8/68 | (12%) | 4/23 | (17%) |
| HBsAg ≤1000 IU/mL and | 8/16 | (50%) | 6/18 | (33%) | 3/14 | (21%) | 3/3 | (100%) |
| HBeAg negative | | | | | | | | |
| HBsAg >1000 IU/mL and | 7/34 | (21%) | 1/29 | (3%) | 2/30 | (7%) | 1/13 | (8%) |
| HBeAg negative | | | | | | | | |
| HBsAg ≤1000 IU/mL and | 0/3 | (0) | 2/5 | (40%) | 2/5 | (40%) | 0/0 | (0) |
| HBeAg positive | | | | | | | | |
| HBsAg >1000 IU/mL and | 1/15 | (7%) | 0/15 | (0) | 1/19 | (5%) | 0/7 | (0) |

HBeAg positive

End of Study

| | | | | | | | | |
|---|---|---|---|---|---|---|---|---|
| Overall | 6/68 | (9%) | 6/67 | (9%) | 2/68 | (3%) | 0/23 | (0) |
| HBsAg ≤1000 IU/mL and | 3/16 | (19%) | 3/18 | (17%) | 1/14 | (7%) | 0/3 | (0) |
| HBeAg negative | | | | | | | | |
| HBsAg >1000 IU/mL and | 2/34 | (6%) | 1/29 | (3%) | 0/30 | (0) | 0/13 | (0) |
| HBeAg negative | | | | | | | | |
| HBsAg ≤1000 IU/mL and | 0/3 | (0) | 2/5 | (40%) | 0/5 | (0) | 0/0 | (0) |
| HBeAg positive | | | | | | | | |
| HBsAg >1000 IU/mL and | 1/15 | (7%) | 0/15 | (0) | 1/19 | (5%) | 0/7 | (0) |

HBeAg positive

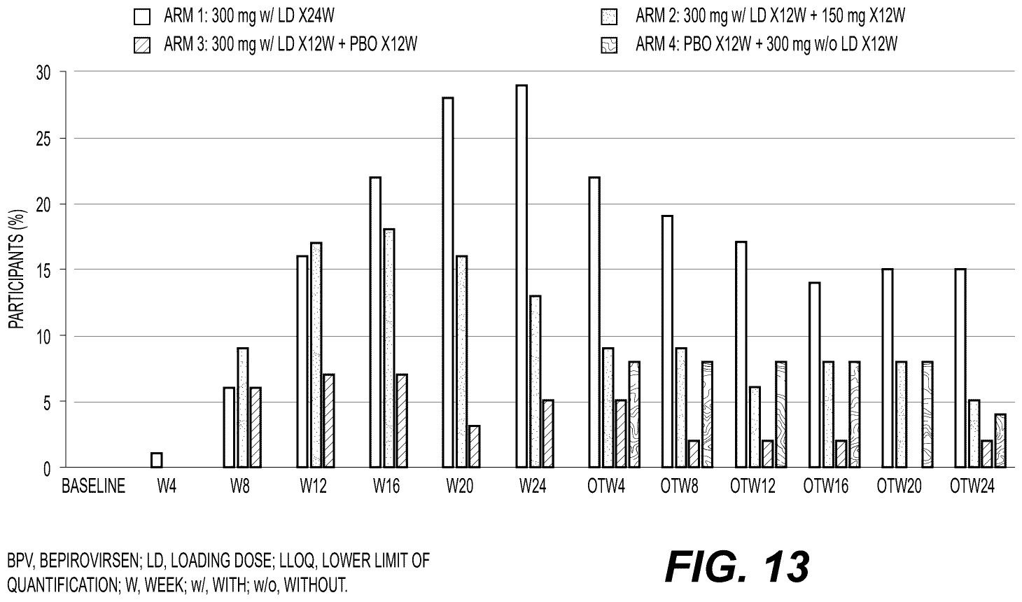

For NOT ON-NUC cohorts, 230 patients (54% male, 57% Asian, 74% HBeAg negative, 19% HBsAg≤3 log 10 IU/mL) were included in the intent-to-treat population (70, 68, 68 and 24 pts, respectively, in arms 1-4). VR up to 24 weeks post-BPV treatment was achieved in 7 (10%), 4 (6%), 1 (1%) and 0 patients in arms 1-4, respectively. VR data over time are shown in FIG. 13. The patients with low baseline HBsAg levels (≤3 log 10 IU/mL) were more likely to have sustained VR (14%) as compared to the patients with high baseline HBsAg levels (>3 log 10 IU/mL; 3%). 12 HBeAg negative patients in arms 1-3 had sustained VR while none of HBeAg positive patients did. 300 mg ×24 weeks BPV (arm 1) resulted in the highest proportion of patients with SVR 24 weeks off BPV treatment. See Table 11.

TABLE 11
Proportion of Participants Achieving End of Treatment Response
and Primary Endpoint at End of Study (Not On-NUC Population)

| | | | |
|---|---|---|---|
| Arm 1 | Arm 2 | Arm 3 | Arm 4 |

End of bepirovirsen treatment

| | | | | | | | | |
|---|---|---|---|---|---|---|---|---|
| Overall | 17/70 | (24%) | 10/68 | (15%) | 6/68 | (9%) | 1/24 | (4%) |
| HBsAg ≤1000 IU/mL and | 7/12 | (58%) | 6/14 | (43%) | 6/10 | (60%) | 1/5 | (20%) |
| HBeAg negative | | | | | | | | |
| HBsAg >1000 IU/mL and | 10/37 | (27%) | 4/38 | (11%) | 0/42 | (0) | 0/12 | (0) |
| HBeAg negative | | | | | | | | |
| HBsAg ≤1000 IU/mL and | 0/0 | (0) | 0/1 | (0) | 0/1 | (0) | 0/0 | (0) |
| HBeAg positive | | | | | | | | |
| HBsAg >1000 IU/mL and | 0/21 | (0) | 0/15 | (0) | 0/15 | (0) | 0/7 | (0) |

HBeAg positive

End of Study

| | | | | | | | | |
|---|---|---|---|---|---|---|---|---|
| Overall | 7/70 | (10%) | 4/68 | (6%) | 1/68 | (1%) | 0/24 | (0) |
| HBsAg ≤1000 IU/mL and | 3/12 | (25%) | 2/14 | (14%) | 1/10 | (10%) | 0/5 | (0) |
| HBeAg negative | | | | | | | | |
| HBsAg >1000 IU/mL and | 4/37 | (11%) | 2/38 | (5%) | 0/42 | (0) | 0/12 | (0) |
| HBeAg negative | | | | | | | | |
| HBsAg ≤1000 IU/mL and | 0/0 | (0) | 0/1 | (0) | 0/1 | (0) | 0/0 | (0) |
| HBeAg positive | | | | | | | | |
| HBsAg >1000 IU/mL and | 0/21 | (0) | 0/15 | (0) | 0/15 | (0) | 0/7 | (0) |

HBeAg positive

### Example 5

Evaluation of the Efficacy and Safety of Treatment with Bepirovirsen in HBeAg-Negative Nucleos(t)ide Anal Ogue-Treated Participants with Chronic Hepatitis B

### A. Study Design

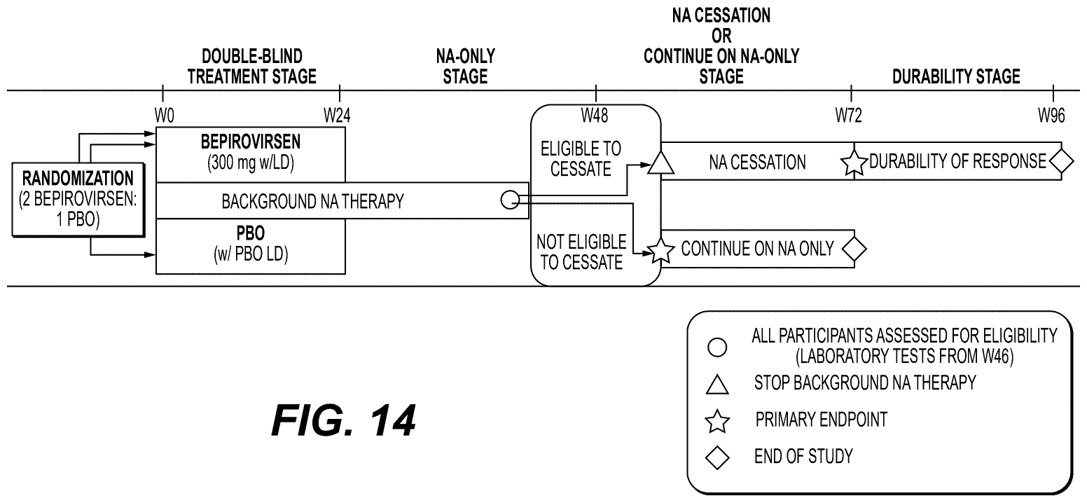

This is a Phase 3, multicenter, randomized, double blind, placebo controlled study to assess efficacy and safety of treatment with bepirovirsen in HBeAg-negative participants with chronic HBV infection on NA treatment. After a 2 part screening-period, this study has 4 stages (FIG. 14):
Double-blind treatment for 24 weeks
NA treatment for 24 weeks
NA cessation with 24 week follow-up OR Continue On-NA for 24 weeks
Durability of response and follow-up for a further 24 weeks for participants who stopped NA treatment at Week 48

The arms will be stratified based on HBsAg level (HBsAg≥100 IU/mL to ≤1000 IU/mL or >1000 IU/mL to ≤3000 IU/mL) at screening.

### B. Objectives and Endpoints

The terms used in this study are defined below:

| Term | Definition |
|---|---|
| Bepirovirsen arm | Bepirovirsen + NA (24 weeks) with loading doses followed by |
| | NA only (24 weeks) then either NA cessation (48 weeks) or |
| | Continue NA (24 weeks) |
| Placebo arm | Placebo + NA (24 weeks) followed by NA only (24 weeks) |
| | then either NA cessation (48 weeks) or Continue NA (24 |
| | weeks) |
| On-NA | On nucleos(t)ide analogue treatment |
| Non-responder | Participant who does not meet NA cessation criteria at Week |
| | 48 and/or does not achieve functional cure (FC) through Week |
| | 72 |
| Functional cure | Sustained suppression (24 weeks or longer) of HBV DNA |
| (clinical response) | (<LLOQ) off all HBV treatment with HBsAg loss |
| | (<0.05 IU/mL) with or without HBsAb after a finite duration |
| | of therapy |
| | FC can only be achieved after NA cessation at W48 |
| HBsAg “blip” | An HBsAg value ≥0.05 IU/mL that is preceded by and |
| | followed by HBsAg values <0.05 IU/mL |
| HBV DNA “blip” | An HBV DNA value ≥LLOQ that is preceded by and followed |
| | by HBV DNA values <LLOQ |
| HBsAg reversion | HBsAg ≥LLOQ at 2 sequential visits |
| HBV DNA loss | HBV DNA <LLOQ at 2 sequential visits |
| HBsAg loss | HBsAg <LLOQ at 2 sequential visits |
| Virologic response | HBsAg <0.05 IU/mL and HBV DNA <LLOQ at W24 |
| Virologic | ≥1.0 log10 IU/mL increase from nadir of HBV DNA OR |
| breakthrough/relapse | HBV DNA becoming quantifiable after being <LLOQ |
| (HBV DNA) | confirmed by 2 sequential visits on-treatment (breakthrough) |
| | or off- treatment (relapse) |
| Post NA Cessation | HBsAg ≥LLOQ and HBV DNA >2000 IU/mL at 2 sequential |
| virologic relapse | visits after cessation of NA treatment. An HBV DNA value |
| | >2000 IU/mL must be confirmed within 1 week (±3 days) |
| | from the date of first result |
| Post NA Cessation | HBsAg ≥LLOQ and HBV DNA >2000 IU/mL and ALT >2 × |
| clinical relapse | ULN at 2 sequential visits after cessation of NA treatment. An |
| | HBV DNA value >2000 IU/mL must be confirmed within 1 |
| | week (±3 days) from the date of first result |

The primary endpoint is: To assess the treatment effect of 24 weeks bepirovirsen with loading doses to achieve functional cure in HBeAg negative participants with chronic HBV infection on NA treatment with baseline HBsAg≤1000 IU/mL.

The key secondary endpoints are:
To assess the treatment effect of 24 weeks bepirovirsen with loading doses to achieve functional cure in HBeAg negative participants with chronic HBV infection on NA treatment with baseline HBsAg≤3000 IU/mL;
To assess the treatment effect of 24 weeks bepirovirsen with loading doses in HBV DNA suppression off-treatment after a finite duration of therapy in HBeAg-negative participants with chronic HBV infection on NA treatment with baseline HBsAg≤1000 IU/mL;
To assess the treatment effect of 24 weeks bepirovirsen with loading doses in HBV DNA suppression after a finite duration of therapy in HBeAg-negative participants with chronic HBV infection on NA treatment with baseline HBsAg 3000 IU/mL.

The safety objective: To assess the safety and tolerability of bepirovirsen when dosed for 24 weeks duration with loading doses in HBeAg-negative participants with chronic HBV infection on NA treatment.

Exploratory objectives and endpoints are:

| Objectives | Endpoints |
|---|---|
| Efficacy: To assess the treatment effect of | Categorical Variables: |
| bepirovirsen on biomarkers and | Achieving HBsAg <0.05 IU/mL and HBV |
| virus-specific antibody responses in | DNA <LLOQ at Week 24 |
| HBeAg-negative participants with chronic | Meeting NA cessation criteria at Week 48 |
| HBV infection on NA treatment | Meeting NA cessation criteria at Week 48 |
| | and sustaining FC for a further 48 weeks |
| | Meeting NA cessation criteria at Week 48 |
| | and sustaining HBV DNA <LLOQ for a |
| | further 48 weeks |
| | Anti-HBeAg status at Weeks 12, 24, 48, |
| | 72, and 96 |
| | Continuous Variables: |
| | Actual values and change from baseline |
| | over time for HBsAg, HBV DNA, and |
| | anti-HBsAg |
| | Correlation between FC and on treatment |
| | biomarkers, including but not limited to, on |
| | treatment HBsAg and change from |
| | baseline |
| Efficacy: To describe virological relapse | Categorical Variables: |
| and clinical relapse after NA cessation in | Meeting virologic relapse and clinical |
| participants who do not achieve FC | relapse criteria |
| | Time to Event Variables: |
| | Time from NA cessation to virological |
| | relapse status in the absence of any rescue |
| | medication |
| | Time from NA cessation to clinical relapse |
| | status in the absence of any rescue |
| | medication |
| Immunogenicity: To investigate the | Incidence of anti-drug antibodies (ADA) at |
| immunogenicity of repeat doses of | specified visits. |
| bepirovirsen | Investigate the effect of ADA on systemic |
| | PK |
| PK-PD relationships: To evaluate | PK, HBsAg and ALT data will be used to |
| PK-efficacy relationship and PK-safety | update the existing population PK and PK-PD |
| relationship | models to ensure the appropriateness of the |
| | dose for the studied population. Relevant |
| | endpoints include: |
| | apparent clearance |
| | apparent volume of distribution |
| | IC50 |
| | random variability |
| | The model will assess the effect of various |
| | factors (covariates) of the modelled endpoints |
| | to confirm that no subpopulation warrants a |
| | dose adjustment |
| Efficacy: To assess the pharmacodynamic | HBV core related antigen (HBcrAg), HBV |
| effect of bepirovirsen on exploratory viral | RNA |
| biomarkers | |
| Virology: To assess the effect of genotype | HBV genotype (using sequencing, serology or |
| and baseline polymorphisms (including | prior investigator reports), and mutation |
| within the bepirovirsen binding site) with | frequency tables from sequencing of the viral |
| treatment response. | HBV DNA and/or HBV RNA prior to |
| To assess the emergence of mutations | treatment, during treatment and post treatment |
| within the bepirovirsen binding site, and | visits |
| elsewhere in the hepatitis B genome, | |
| during and after treatment | |
| Immunology and Disease: To assess the | Correlation between the following: |
| pharmacodynamic effect of bepirovirsen | Virological biomarkers, as determined by |
| on immunological and disease biomarkers. | (but not limited to) specific viral |
| To describe the relationship(s) between | parameters (HBsAg, HBV RNA, HBV |
| virology biomarkers, including but not | DNA, HBcrAg) |
| limited to HBsAg, disease and | Soluble immunological and disease |
| immunological biomarkers | biomarkers, as determined by (but not |
| | limited to) levels of circulating cytokines |
| | and chemokines and markers of fibrosis. |
| | Markers of immune cell function, which |
| | may include relative frequencies of |
| | immune cell subsets among PBMCs, |
| | activation status as determined by |
| | phenotyping and gene expression patterns, |
| | and functional assays including |
| | HBV-specific cytokine and/or antibody |
| | production |
| Patient Reported Outcomes: To assess | Change from baseline of 8.10.1. Hepatitis B |
| changes from baseline in patient reported | Quality of Life (HBQOL) and EQ-5D-3L (a |
| outcomes following treatment with | family of instruments to describe and value |
| bepirovirsen | health) at Week 24, Week 48, and Week 72, |
| | Week 96 |

### C. Study Population: Inclusion and Exclusion Criteria

The inclusion criteria are:
1. At least 18 years of age at the time of signing the informed consent (if country/site age requirements for consent differ, the more stringent [e.g., higher age] restriction will be required for that country/site).
2. Participants must be HBeAg negative at screening.
3. Participants who have documented chronic HBV infection ≥6 months prior to Screening AND
   Currently receiving stable NA therapy defined as no changes to their NA regimen from at least 6 months prior to Screening and with no planned changes to the stable regimen over the duration of the study
4. Plasma or serum HBsAg concentration>100 IU/mL, but no greater than 3000 IU/mL.
5. Plasma or serum HBV DNA concentration must be adequately suppressed, defined as plasma or serum HBV DNA≤90 IU/mL.
6. Alanine aminotransferase (ALT)≤2×ULN.
7. Participants who are willing and able to cease their NA treatment in accordance with the protocol.

The exclusion criteria are:

### Medical Conditions

1. Clinically significant abnormalities, aside from chronic HBV infection in medical history (e.g., moderate-severe liver disease other than chronic HBV, acute coronary syndrome within 6 months of screening, major surgery within 3 months of screening, significant/unstable cardiac disease, uncontrolled diabetes, bleeding diathesis or coagulopathy) or physical examination
2. Co-infection with:
   a. Current history of Hepatitis C infection or participants that have been cured for <12 months at the time of screening
   b. Human immunodeficiency virus (HIV)
   c. Hepatitis D virus
3. History of or suspected liver cirrhosis and/or evidence of cirrhosis as determined by
   a. Both APRI>2 and FibroSure/FibroTest result>0.7
      If only 1 parameter (APRI or FibroSure/FibroTest) result is positive, a discussion with the Medical Monitor is required before inclusion in study is permitted
   b. Regardless of APRI of Fibrosure/FibroTest score, if the participant meets 1 of the following criteria documented from their medical history, they will be excluded from the study
      Liver biopsy (i.e., Metavir Score F4)
      Liver stiffness >12 kPa 4. Diagnosed or suspected hepatocellular carcinoma as evidenced by the following
   a. Alpha-fetoprotein concentration≥200 ng/mL
   b. If the screening alpha fetoprotein concentration is ≥50 ng/mL and <200 ng/mL, the absence of liver mass must be documented by imaging within 6 months before randomization
5. History of malignancy within the past 5 years with the exception of specific cancers that are cured by surgical resection (e.g., skin cancer). Participants under evaluation for possible malignancy are not eligible.
6. History of vasculitis or presence of symptoms and signs of potential vasculitis (e.g., vasculitic rash, skin ulceration, repeated blood detected in urine without identified cause) or history/presence of other diseases that may be associated with vasculitis condition (e.g., systemic lupus erythematosus, rheumatoid arthritis, relapsing polychondritis, mononeuritis multiplex)
7. History of extrahepatic disorders possibly related to HBV immune conditions (e.g., nephrotic syndrome, any type of glomerulonephritis, polyarteritis nodosa, cryoglobulinemia, uncontrolled hypertension)
8. History of alcohol or drug abuse/dependence
   a. Current alcohol use as judged by investigator to potentially interfere with participant compliance
   b. History of or current drug abuse/dependence as judged by the investigator to potentially interfere with participant compliance
      i. Refers to illicit drugs and substances with abuse potential. Medications that are used by the participant as directed, whether over-the-counter or through prescription, are acceptable and would not meet the exclusion criteria

### Prior/Concomitant Therapy

9. Currently taking, or took within 3 months of screening, any immunosuppressing drugs (e.g., prednisone), other than a short course of therapy (≤2 weeks) or topical/inhaled steroid use.
10. Participants to whom immnosuppressive treatment, including therapeutic doses of steroids is contraindicated, should not be considered for enrolment in the study.
11. Currently taking, or HAS TAKEN within 12 months of Screening, any interferon-containing therapy.
12. Participants requiring anti-coagulation therapies (e.g., warfarin, Factor Xa inhibitors) or anti-platelet agents (like clopidogrel or aspirin) unless treatment can safely be discontinued throughout duration of the study, by the discretion of the investigator. Occasional use is permitted.
13. The participant has participated in a clinical trial and has received an investigational product within the following time period prior to the first dosing day in the current study: 5 half-lives (if known) or twice the duration (if known) of the biological effect of the study treatment (whichever is longer) or 90 days (if half-life or duration is unknown).
14. Prior treatment with any oligonucleotide or siRNA within 12 months prior to the first dosing day (excluding COVID vaccinations).
15. Prior treatment with bepirovirsen.

### Diagnostic Assessments

16. Fridericia's QT correction formula (QTcF) ≥450 msec (if single ECG at screening shows QTcF 450 msec, a mean of triplicate measurements should be used to confirm that participant meets exclusion criterion).
17. Laboratory results as follows:
   a. Serum albumin ≤3.5 g/dL
   b. Glomerular filtration rate (GFR) 60 mL/min/1.73 m² as calculated by the CKD-EPI formula (for Japan, JSN-CKDI equation)
   c. International normalized ratio (INR) >1.25
   d. Platelet count <140×10⁹/L
e. Total bilirubin >1.25×ULN
   For participants with benign unconjugated hyperbilirubinemia with total bilirubin >1.25×ULN, discussion for inclusion to the study is required with the Medical Monitor
f. Urine albumin to creatinine ratio (uACR) >0.3 mg/mg (or >300 mg/g). In the event of an uACR above this threshold, eligibility may be confirmed by a second measurement
   In cases where participants have low urine albumin and low urine creatinine levels resulting in a uACR calculation >0.3 mg/mg (or >300 mg/g), the investigator should confirm that the participant does not have a history of diabetes, hypertension or other risk factors that may affect renal function and discuss with the Medical Monitor, or designee

### Other Exclusions

18. History of/sensitivity to bepirovirsen or components thereof or a history of drug or other allergy that, in the opinion of the investigator or Medical Monitor, contraindicates their participation
19. Participants who do not wish to discontinue taking NA therapy for their chronic HBV infection.

### D. Study Assessments and Procedures

The primary efficacy endpoint is functional cure, as defined herein. The primary objective measurements for efficacy include HBsAg and HBV DNA.

Any detectable HBsAg or HBV DNA after achieving HBsAg<0.05 IU/mL and HBV DNA<LLOQ needs to be confirmed by re-test within 1 week (±3 days) of receiving the test result. At Week 96 (end of study visit), no retest is required.

Safety assessments are conducted at planned time points during the course of the study, and additional time points for safety tests may be added based on newly available data to ensure appropriate safety monitoring. Safety assessments include physical examinations, injection site reactions, vital signs, electrocardiograms, and clinical safety laboratory assessment.

Adverse events (AEs) and serious adverse events (SAEs) are detected, documented, and reported. Adverse Events of special interest include: ALT increase (flares), vascular inflammation and complement activation, thrombocytopenia, and renal injury.

Blood samples are collected for measurement of plasma concentrations of bepirovirsen.

Pharmacodynamic parameters will include but are not limited to:
Categorical: virologic response, seroclearance (HBsAg), HBV DNA<LLOQ, and seroconversion (HBsAb);
Change from baseline: HBsAg, HBV DNA, and HBsAb levels.
Safety assessments including, but not limited to, vital signs, laboratory measurements and AEs.

Collection of samples for other biomarker research is also part of this study. These exploratory biomarker samples will be collected to evaluate the pathogenesis of chronic HBV infection; the absorption, distribution, metabolism, or excretion of bepirovirsen; or the participant's response to bepirovirsen. In addition, continuing research may identify other proteins, transcripts, biomarkers, or assays related to bepirovirsen treatment, the response to bepirovirsen or the pathogenesis of chronic HBV infection, which will be evaluated in these samples.

### Example 6

### Amendment to the Phase 3 Study of Bepirovirsen in Nucleos(t)Ide Analogue-Treated Participants with Chronic Hepatitis B

The amendment was made to include participants with HBeAg positive chronic HBV infection, to increase the frequency of monitoring post NA cessation, and to switch the primary and secondary endpoints.

Approximately 750 to 1050 participants will be randomly assigned to 24 weeks of treatment with either bepirovirsen or placebo (2:1 ratio). The planned minimum sample size of 750 provides 99% power for the primary endpoint of functional cure in the baseline HBsAg≤3000 IU/mL population and 98% power for the key secondary endpoint of functional cure in the baseline HBsAg≤1000 IU/mL populations.

The primary endpoint is: To assess the treatment effect of 24 weeks bepirovirsen with loading doses to achieve functional cure in participants with chronic HBV infection on NA treatment with baseline HBsAg 3000 IU/mL.

The key secondary endpoints are:
To assess the treatment effect of 24 weeks bepirovirsen with loading doses to achieve functional cure in participants with chronic HBV infection on NA treatment with baseline HBsAg≤1000 IU/mL;
To assess the treatment effect of 24 weeks bepirovirsen with loading doses in HBV DNA suppression off-treatment after a finite duration of therapy in participants with chronic HBV infection on NA treatment with baseline HBsAg≤3000 IU/mL;
To assess the treatment effect of 24 weeks bepirovirsen with loading doses in HBV DNA suppression off-treatment after a finite duration of therapy in participants with chronic HBV infection on NA treatment with baseline HBsAg≤1000 IU/mL.

The safety objective: To assess the safety and tolerability of bepirovirsen when dosed for 24 weeks duration with loading doses in participants with chronic HBV infection on NA treatment.

One skilled in the art will readily appreciate that the present disclosure is well adapted to carry out the objects and obtain the ends and advantages mentioned, as well as those inherent therein. The particular embodiments described herein are intended to be representative and exemplary and are not intended as limitations on the scope of the invention. Changes therein and other uses will be apparent to those skilled in the art which are encompassed within the spirit of the invention as defined by the scope of the claims.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method for treating chronic hepatitis B in a human in need thereof, the method comprising administering to the human a therapeutically effective amount of bepirovirsen, wherein the human has a HBsAg baseline level of not greater than a threshold level.

2. (canceled)

3. (canceled)

4. (canceled)

5. (canceled)

6. The method of claim 1, wherein the threshold level is about 1000 IU/mL.

7. The method of claim 1, wherein the threshold level is about 3000 IU/mL.

8. The method of claim 1, wherein the human is not treated with another HBsAg reducing agent or immunomodulating agent.

9. The method of claim 1, wherein bepirovirsen is administered by subcutaneous injection.

10. The method of claim 1, wherein bepirovirsen is administered at a dose of about 300 mg once weekly.

11. The method of claim 1, wherein bepirovirsen is administered for 12 weeks to 48 weeks.

12. The method of claim 10, wherein bepirovirsen is administered weekly with additional loading doses in the first two weeks on Day 4 and Day 11 following the first dose.

13. The method of claim 1, wherein the human is on stable nucleoside or nucleotide analogue (NA) therapy.

14. The method of claim 13, wherein the NA therapy is lamivudine, adefovir, adefovir dipivoxil, telbivudine, entecavir, tenofovir, tenofovir disoproxil fumarate, or tenofovir alafenamide, or a pharmaceutically acceptable salt thereof.

15. The method of claim 1, wherein the human is not treated with a nucleoside or nucleotide analogue (NA).

16. The method of claim 1, wherein the human is HBeAg negative prior to the bepirovirsen treatment.

17. The method of claim 1, wherein the human is HBeAg positive prior to the bepirovirsen treatment.

18. (canceled)

19. (canceled)

20. The method of claim 1, wherein the threshold level is about 3000 IU/mL, wherein bepirovirsen is administered at a dose of about 300 mg once weekly for 24 weeks with additional loading doses in the first two weeks on Day 4 and Day 11 following the first dose.

21. The method of claim 20, wherein the human is on stable nucleoside or nucleotide analogue (NA) therapy.

22. The method of claim 21, wherein the NA therapy is lamivudine, adefovir, adefovir dipivoxil, telbivudine, entecavir, tenofovir, tenofovir disoproxil fumarate, or tenofovir alafenamide, or a pharmaceutically acceptable salt thereof.
